(19) United States
(12) Patent Application Publication
(10) Pub. No.: US 2023/0405094 A1
(43) Pub. Date: Dec. 21, 2023

(54) TREATMENT OR PREVENTION OF A DISEASE OR DISORDER

(71) Applicant: Janssen Sciences Ireland Unlimited Company, Co Cork (IE)

(72) Inventors: René Holm, Woluwe Saint Pierre (BE); Steven M. Vesole, Redwood City, CA (US); Guenter Karl Wilhelm Kraus, Frankfurt AM Main (DE)

(21) Appl. No.: 18/037,218

(22) PCT Filed: Nov. 17, 2021
(86) PCT No.: PCT/EP2021/082031

§ 371 Date: May 16, 2023

Related U.S. Application Data
(60) Provisional application No. 63/115,002, filed on Nov. 17, 2020.

(51) Int. Cl. A61K 38/47; A61K 9/00; A61K 9/10; A61K 31/505; A61K 9/16; A61P 31/18

(52) U.S. Cl. CPC A61K 38/47; A61K 9/0019; A61K 9/10; A61K 31/505; A61K 9/1641; A61K 9/1623; A61P 31/18; C12Y 302/01035

(57) ABSTRACT

The present invention relates to the treatment or prevention of a disease or disorder using a drug in the form of micro- or nanoparticles in suspension, in combination with a hyaluronidase.

### TECHNICAL FIELD

The present invention relates to the treatment or prevention of a disease or disorder using a drug in the form of micro- or nanoparticles in suspension, in combination with a hyaluronidase.

### BACKGROUND AND RELATED ART

It is often desirable to keep the blood plasma concentration of a drug above a minimum level in order for the drug to provide an effective treatment or prevention of a disease or disorder. At blood plasma concentrations lower than the minimum blood plasma level, the drug may no longer be effective.

The time interval between administrations of a drug may be selected to alter the blood plasma levels of the drug and its metabolites. The time interval may be short (e.g. one day) where the drug reaches the blood plasma quickly and does not remain in the blood plasma for a long time period, or the time interval may be long (e.g. six months) where slower release into the blood plasma or slower clearance from the blood plasma means that the blood plasma levels of the drug are sufficiently high for a long time period.

Because of their pharmacokinetic properties and the need to keep blood plasma levels above a minimum level, many drugs require frequent administration, often at high doses. The number and/or volume of the dosage forms containing the drug that needs to be administered is commonly referred to as the “pill burden”. While a high pill burden may enable the blood plasma level to be kept suitably high, it is undesirable for many reasons.

For example, a high pill burden requires a high frequency of intake and often large volumes of the dosage form need to be stored and transported. For drugs administered by subcutaneous or intramuscular injection, patient tolerability is an additional concern, certainly when large volumes are injected. For example, administration by subcutaneous or intramuscular injection can result in irritation, inflammation, swelling, acute pain and/or redness and bruising during and after injection at the injection site (injection site reactions). Subcutaneous and intramuscular injections may also be associated with the manifestation of a bump at the surface of the skin at the injection site. Such effects are generally exaggerated by a high injection volume. Such a bump may reveal that the subject concerned received a high volume injection and may hence reveal that the subject is receiving an intervention for a disease or disorder.

Therefore, there is a need to provide drug formulations that allow the blood plasma level of the administered drug to remain above the minimum level for treatment or prevention, thus allowing intermittent administration at longer time periods, and which are well tolerated, which in turn improves patient compliance. There is also a need for this method to be non-visible to the outside world. Areas of interest include but are not limited to the treatment of human immunodeficiency virus (HIV) infection, and the treatment of cancer.

### SUMMARY OF THE INVENTION

In a first aspect there is provided a method for the treatment or prevention of a disease or disorder in a subject in need thereof, the method comprising administering to the subject a drug effective in the treatment or prevention of the disease or disorder in the form of micro- or nanoparticles in suspension by intramuscular injection or subcutaneous injection, wherein the drug is administered in combination with a hyaluronidase that is administered by intramuscular injection or subcutaneous injection, and wherein the drug and the hyaluronidase are administered intermittently at a time interval of about three months to about two years.

In a second aspect there is provided a drug and a hyaluronidase for use in therapy, wherein the drug is in the form of micro- or nanoparticles in suspension, wherein the drug and hyaluronidase are administered by intramuscular injection or subcutaneous injection, and wherein the drug and hyaluronidase are administered intermittently at a time interval of about three months to about two years.

In a third aspect there is provided products containing a drug and a hyaluronidase as a combined preparation for simultaneous or sequential use in therapy by intramuscular injection or subcutaneous injection, wherein the drug is in the form of micro- or nanoparticles in suspension, and wherein the drug and the hyaluronidase are administered intermittently at a time interval of about three months to about two years.

In a fourth aspect there is provided a kit of parts comprising a drug and a hyaluronidase for simultaneous or sequential use in therapy by intramuscular injection or subcutaneous injection, wherein the drug is in the form of micro- or nanoparticles in suspension, and wherein the drug and the hyaluronidase are administered intermittently at a time interval of about three months to about two years.

In a fifth aspect there is provided a drug in the form of micro- or nanoparticles in suspension for use in therapy by intramuscular or subcutaneous injection, wherein the drug is administered in combination with a hyaluronidase that is administered by intramuscular injection or subcutaneous injection, and wherein the drug and the hyaluronidase are administered intermittently at a time interval of about three months to about two years.

In a sixth aspect there is provided use of a drug for the manufacture of a medicament for use in the treatment of a disease or disorder in a subject, wherein the drug is in the form of micro- or nanoparticles in suspension and is administered in combination with a hyaluronidase, wherein the drug and the hyaluronidase are administered to the subject by subcutaneous or intramuscular injection, and wherein the drug and the hyaluronidase are administered intermittently at a time interval of about three months to about two years.

Administration of the drug with a hyaluronidase improves patient tolerability for subcutaneous or intramuscular injection administration routes compared with subcutaneous or intramuscular injection administration of the drug alone, in particular when large volumes are injected. The hyaluronidase may facilitate a more rapid administration of the drug as it may lower the resistance of the tissue against which the drug suspension is delivered. The hyaluronidase may reduce leakage of the drug from the site of injection by decreasing the tissue backpressure. The hyaluronidase may also allow for delivery of larger volumes in patients with less subcutaneous tissue (or lower body mass index). The hyaluronidase may allow the use of a shorter needle.

In addition, it has surprisingly been found that an extended, sustained or prolonged release of a drug into the blood plasma achieved by intramuscular injection or subcutaneous injection of a suspension of micro- or nanoparticles can be maintained when the drug is administered by intramuscular injection or subcutaneous injection with a hyaluronidase as defined herein. As discussed in more detail below in the section titled “Hyaluronidase”, hyaluronidases are used for increasing the dispersion and absorption of injected active pharmaceutical ingredients. In view of this, it is surprising that the inventors have demonstrated that administration of a hyaluronidase with a drug maintains an extended, sustained or prolonged release of drug into the bloodstream.

### BRIEF DESCRIPTION OF THE FIGURES

The invention will be described, by way of example only, with reference to the accompanying figures.

FIG. 1: Mean plasma concentration over time following administration of a drug nanosuspension and a hyaluronidase according to the invention and of a drug nanosuspension alone.

FIG. 2: Mean plasma concentration over six months following administration of a rilpivirine nanosuspension and hyaluronidase according to the invention and of a rilpivirine nanosuspension alone.

These figures are explained further in the “Examples” section.

### DISCLOSURE OF THE INVENTION

This application has been drafted in sections to aid readability. However, this does not mean that each section is to be read in isolation. To the contrary, unless otherwise specified, each section is to be read with cross-referencing to the other sections, i.e. taking the entire application as a whole. No artificial separation of embodiments is intended, unless explicitly stated.

Thus, all of the embodiments described herein relating to the first aspect of the invention apply equally to, i.e. are also disclosed in relation to/combination with, the other aspects described herein.

### DETAILED DESCRIPTION OF THE INVENTION

The Drug(s) Used in the Invention

The drug used in the invention as described herein is in suspension. Preferably, the drug used in the invention is in the form of micro- or nanoparticles in suspension, i.e. a suspension of the drug, wherein the drug is in the form of microparticles or nanoparticles, in particular microparticles or nanoparticles of the drug suspended in a pharmaceutically acceptable carrier, such as for example a pharmaceutically acceptable aqueous carrier. For the avoidance of doubt, the drug described herein is not the hyaluronidase described herein.

The skilled person would understand that the size of the micro- or nanoparticles should be below a maximum size above which administration by subcutaneous or intramuscular injection becomes impaired or is even no longer possible. The maximum size depends for example on the limitations imposed by the needle diameter or by adverse reactions of the body to large particles, or both.

In a preferred embodiment, the drug is in the form of nanoparticles.

In an embodiment, the micro- or nanoparticles described herein have an average effective particle size of less than about 20 μm. In an embodiment, the micro- or nanoparticles have an average effective particle size of less than about 10 μm. In an embodiment, the micro- or nanoparticles have an average effective particle size of less than about 5 μm. In an embodiment, the micro- or nanoparticles have an average effective particle size of less than about 1 μm. In an embodiment, the micro- or nanoparticles have an average effective particle size of less than about 500 nm.

In another embodiment, the micro- or nanoparticles described herein have an average effective particle size of from about 25 nm to about 20 μm. In another embodiment, the micro- or nanoparticles have an average effective particle size of from about 25 nm to about 10 μm (e.g. about 200 nm to about 10 μm). In another embodiment, the micro- or nanoparticles have an average effective particle size of from about 25 nm to about 5 μm (e.g. about 200 nm to about 5 μm). In another embodiment, the micro- or nanoparticles have an average effective particle size of from about 25 nm to about 1 μm. In another embodiment, the micro- or nanoparticles have an average effective particle size of from about 25 nm to about 500 nm, e.g. about 100 nm to about 300 nm.

When the drug is rilpivirine or a pharmaceutically acceptable salt thereof the micro- or nanoparticles preferably have an average effective particle size of from about 100 nm to about 300 nm, for example about 150 nm to about 250 nm or about 180 nm to about 220 nm, e.g. about 200 nm.

The term “average effective particle size” as used herein refers to the volume-based median particle diameter (Dᵥ50), i.e. the diameter below which 50% by volume of the particle population is found.

The average effective particle sizes, i.e. the volume-based median particle diameter, as used herein are determined by routine laser diffraction techniques, e.g. in accordance with ISO 13320:2009.

Laser diffraction relies on the principle that a particle will scatter light at an angle that varies depending on the size the particle and a collection of particles will produce a pattern of scattered light defined by intensity and angle that can be correlated to a particle size distribution. A number of laser diffraction instruments are commercially available for the rapid and reliable determination of particle size distributions. For example, particle size distribution may be measured by the conventional Malvern Mastersizer™ 3000 particle size analyzer from Malvern Instruments. The Malvern Mastersizer™ 3000 particle size analyzer operates by projecting a helium-neon gas laser beam through a transparent cell containing the particles of interest suspended in an aqueous solution. Light rays which strike the particles are scattered through angles which are inversely proportional to the particle size and a photodetector array measures the intensity of light at several predetermined angles and the measured intensities at different angles are processed by a computer using standard theoretical principles to determine the particle size distribution. Laser diffraction values may be obtained using a wet dispersion of the particles in distilled water.

Other methods that are commonly used in the art to measure volume-based median particle diameters include disc centrifugation, scanning electron microscopy (SEM), sedimentation field flow fractionation and photon correlation spectroscopy.

In an embodiment, the method or use or combination or products or kit of parts as described herein are used in combination with one or more other drugs.

In an embodiment, said one or more other drugs is administered at the same intermittent time interval as the drug and hyaluronidase as described herein, e.g. the drug, hyaluronidase and the other drug are administered intermittently at a time interval of about three months, or of about four months, or of about five months or of about six months or of about seven months or of about eight months or of about ten months or of about eleven months or of about one year or of about one year to about 2 years. In an embodiment the drug, the hyaluronidase and the one or more other drugs are administered simultaneously or sequentially by intramuscular or subcutaneous injection, in particular subcutaneous injection. In an embodiment the drug, the hyaluronidase and the one or more other drugs, are administered simultaneously, in particular by subcutaneous injection. In an embodiment the drug, the hyaluronidase and the one or more other drugs, are administered sequentially, in particular by subcutaneous injection. In an embodiment, the hyaluronidase is administered first followed by sequential administration of the drugs.

In an embodiment, more than one drug in the form of micro- or nanoparticles in suspension is used in the invention.

The term “drug” includes any substance that is biologically active, for example a compound in free base form or a pharmaceutically acceptable salt thereof, and also encompasses tautomers, solvates (e.g. hydrates) and crystalline or amorphous solid forms thereof, and the like. The term “drug” also includes prodrugs. In an embodiment, the drug is not a biologic. By “biologic” it is meant a virus, therapeutic serum, toxin, antitoxin, vaccine, blood, blood component or derivative, allergenic product, protein, or analogous product, or arsphenamine or derivative of arsphenamine (or any other trivalent organic arsenic compound), applicable to the prevention, treatment, or cure of a disease or condition, for example, of human beings. In an embodiment, the drug is not an antibody. In another embodiment, the drug has a molecular weight (MW) of less than 1000 Da. In another embodiment, the drug has a molecular weight (MW) of less than 1000 Da and is not a biologic.

In an embodiment, the drug is selected from drugs for treatment of chronic and long-term diseases and disorders, for example for treatment of chronic viral infection (such as chronic infection with Varicella-zoster virus, measles virus, HIV, hepatitis B virus, hepatitis C virus, hepatitis D virus or human cytomegalovirus), cancer, psychiatric diseases and disorders, mood disorders (such as bipolar, cyclothymic or depression), diabetes, hypertension, abnormal cholesterol and triglyceride levels, inflammatory disorders (such as allergy, asthma, autoimmune diseases, coeliac disease, hepatitis, inflammatory bowel disease, Crohn disease, gout, myositis, scleroderma, rheumatoid arthritis, lupus vasculitis, ankylosing spondylitis or chronic obstructive pulmonary disease), cystic fibrosis, multiple sclerosis, autoimmune disorders, neurodegenerative disorders (such as Parkinson Disease or Alzheimer disease), chronic pain, inherited metabolic disorders or epilepsy. In a preferred embodiment, the drug is selected from the list consisting of rilpivirine (TMC278), cabotegravir, apalutamide, enzalutamide, and darolutamide, or pharmaceutically acceptable salts thereof.

In an embodiment, the drug is rilpivirine or a pharmaceutically acceptable salt thereof, in particular rilpivirine.

Rilpivirine (4-[[4-[[4-[(1E)-2-cyanoethenyl]-2,6-dimethylphenyl]amino]-2-pyrimidinyl]amino]benzonitrile; TMC278) has the following structural formula:

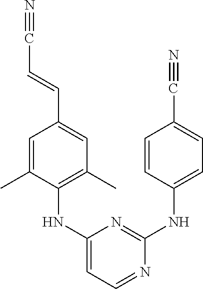 [SMILES: Cc1cc(/C=C/C#N)cc(C)c1Nc1ccnc(Nc2ccc(C#N)cc2)n1]

By “rilpivirine” it is meant rilpivirine having the structural formula shown above, i.e. the free base form.

Pharmaceutically acceptable salts of rilpivirine means those where the counterion is pharmaceutically acceptable. The pharmaceutically acceptable salts are meant to comprise the therapeutically active non-toxic acid addition salt forms which rilpivirine is able to form. These salt forms can conveniently be obtained by treating rilpivirine with such appropriate acids as inorganic acids, for example, hydrohalic acids, e.g. hydrochloric, hydrobromic and the like; sulfuric acid; nitric acid; phosphoric acid and the like; or organic acids, for example, acetic, propanoic, hydroxyacetic, 2-hydroxypropanoic, 2-oxopropanoic, oxalic, malonic, succinic, maleic, fumaric, malic, tartaric, 2-hydroxy-1,2,3-propanetricarboxylic, methanesulfonic, ethanesulfonic, benzenesulfonic, 4-methylbenzene sulfonic, cyclohexanesulfamic, 2-hydroxybenzoic, 4-amino-2-hydroxybenzoic and the like acids.

In an embodiment, the drug described herein is a next-generation anti-androgen. In an embodiment, the drug is apalutamide.

As used herein, the term “next-generation anti-androgen” refers to an agent that exhibits full antagonist activity against a wild-type androgen receptors (AR) polypeptide. Next-generation anti-androgens differ from first-generation anti-androgens in that second-generation anti-androgens act as full antagonists in cells expressing elevated levels of AR, such as for example, in castration resistant prostate cancers (CRPC).

Exemplary next-generation anti-androgens described herein include apalutamide, enzalutamide (CAS No: 915087-33-1), RD162 (CAS No. 915087-27-3) and darolutamide.

In some embodiments, the next-generation anti-androgen described herein binds to an AR polypeptide at or near the ligand binding site of the AR polypeptide.

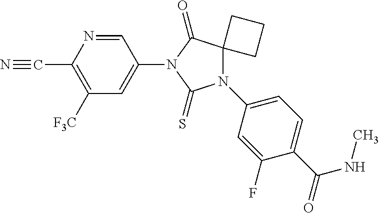 [SMILES: CNC(=O)c1ccc(N2C(=S)N(c3cnc(C#N)c(C(F)(F)F)c3)C(=O)C23CCC3)cc1F]

### 4-[7-(6-cyano-5-trifluoromethylpyridin-3-yl)-8-oxo-6-thioxo-5,7-diazaspiro[3.4]oct-5-yl]-2-fluoro-N-methylbenzamide (apalutamide)

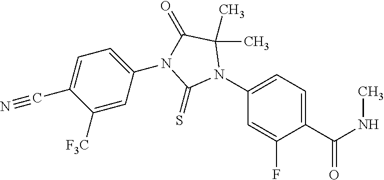 [SMILES: CNC(=O)c1ccc(N2C(=S)N(c3ccc(C#N)c(C(F)(F)F)c3)C(=O)C2(C)C)cc1F]

### 4-(3-(4-cyano-3-(trifluoromethyl)phenyl)-5,5-dimethyl-4-oxo-2-thioxoimidazolidin-1-yl)-2-fluoro-N-methylbenzamide (enzalutamide)

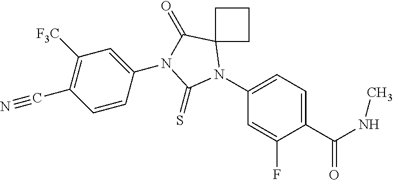 [SMILES: CNC(=O)c1ccc(N2C(=S)N(c3ccc(C#N)c(C(F)(F)F)c3)C(=O)C23CCC3)cc1F]

### 4-[7-(4-cyano-3-trifluoromethylphenyl)-8-oxo-6-thioxo-5,7-diazaspiro[3.4]oct-5-yl]-2-fluoro-N-methylbenzamide (RD162)

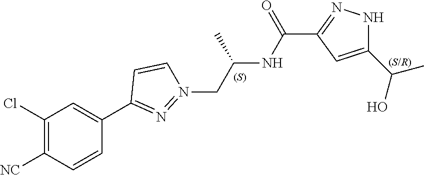

### N-{(2S)-1-[3-(3-chloro-4-cyanophenyl)-1H-pyrazol-1-yl]propan-2-yl}-5-(1-hydroxyethyl)-1H-pyrazole-3-carboxamide (darolutamide)

In some embodiments, an anti-androgen contemplated in the aspects of the invention described herein inhibits AR nuclear translocation, such as darolutamide, DNA binding to androgen response elements, and coactivator recruitment. In some embodiments, an anti-androgen contemplated in the aspects of the invention described herein exhibits no agonist activity in AR-overexpressing prostate cancer cells.

Apalutamide is a next-generation anti-androgen that binds directly to the ligand-binding domain of AR, impairing nuclear translocation, AR binding to DNA and AR target gene modulation, thereby inhibiting tumor growth and promoting apoptosis. Apalutamide binds AR with greater affinity than bicalutamide and induces partial or complete tumor regression in non-castrate hormone-sensitive and bicalutamide-resistant human prostate cancer xenograft models (Clegg et al. Cancer Res. Mar. 15, 2012 72; 1494). Apalutamide lacks the partial agonist activity seen with bicalutamide in the context of AR overexpression.

Darolutamide, BAY1841788 or ODM-201, is an AR antagonist that includes two diastereomers—ORM-16497 and ORM-16555. It has activity against known AR mutants that confer resistance to other second-generation anti-androgens. Darolutamide binds to the AR with high affinity and impairs subsequent androgen-induced nuclear translocation of AR and transcription of AR gene target (Matsubara, N., Mukai, H., Hosono, A. et al. Cancer Chemother Pharmacol (2017) 80: 1063).

In an embodiment, the micro- or nanoparticles have one or more surface modifiers adsorbed to their surface.

The surface modifier may be selected from known organic and inorganic pharmaceutical excipients, including various polymers, low molecular weight oligomers, natural products and surfactants. Particular surface modifiers that may be used in the invention include nonionic and anionic surfactants. Representative examples of surface modifiers include gelatin, casein, lecithin, salts of negatively charged phospholipids or the acid form thereof (such as phosphatidyl glycerol, phosphatidyl inosite, phosphatidyl serine, phosphatic acid, and their salts such as alkali metal salts, e.g. their sodium salts, for example egg phosphatidyl glycerol sodium, such as the product available under the tradename Lipoid™ EPG), gum acacia, stearic acid, benzalkonium chloride, polyoxyethylene alkyl ethers, e.g., macrogol ethers such as cetomacrogol 1000, polyoxyethylene castor oil derivatives; polyoxyethylene stearates, colloidal silicon dioxide, sodium dodecylsulfate, carboxymethylcellulose sodium, bile salts such as sodium taurocholate, sodium desoxytaurocholate, sodium desoxycholate; methylcellulose, hydroxyethylcellulose, hydroxypropylcellulose, hydroxypropylmethylcellulose, magnesium aluminate silicate, polyvinyl alcohol (PVA), poloxamers, such as Pluronic™ F68, F108 and F127 which are block copolymers of ethylene oxide and propylene oxide; tyloxapol; Vitamin E-TGPS (α-tocopheryl polyethylene glycol succinate, in particular α-tocopheryl polyethylene glycol 1000 succinate); poloxamines, such as Tetronic™ 908 (T908) which is a tetrafunctional block copolymer derived from sequential addition of ethylene oxide and propylene oxide to ethylenediamine; dextran; lecithin; dioctyl ester of sodium sulfosuccinic acid such as the products sold under the tradename Aerosol OT™ (AOT); sodium lauryl sulfate (Duponol™ P); alkyl aryl polyether sulfonate available under the tradename Triton™ X-200; polyoxyethylene sorbitan fatty acid esters (Tweens™ 20, 40, 60 and 80); sorbitan esters of fatty acids (Span™ 20, 40, 60 and 80 or Arlacel™ 20, 40, 60 and 80); polyethylene glycols (such as those sold under the tradename Carbowax™ 3550 and 934); sucrose stearate and sucrose distearate mixtures such as the product available under the tradename Crodesta™ F110 or Crodesta™ SL-40; hexyldecyl trimethyl ammonium chloride (CTAC); polyvinylpyrrolidone (PVP). If desired, two or more surface modifiers can be used in combination.

In an embodiment, the surface modifier is selected from a poloxamer, α-tocopheryl polyethylene glycol succinate, polyoxyethylene sorbitan fatty acid ester, and salts of negatively charged phospholipids or the acid form thereof. In a preferred embodiment, the surface modifier is selected from Pluronic™ F108, Vitamin E TGPS, Tween™ 80, and Lipoid™ EPG.

Pluronic™ F108 corresponds to poloxamer 338 and is the polyoxyethylene, polyoxypropylene block copolymer that conforms generally to the formula HO—[CH₂CH₂O]r-[CH(CH₃)CH₂O]_{y}—[CH₂CH₂O]_{z}—H in which the average values of x, y and z are respectively 128, 54 and 128. Other commercial names of poloxamer 338 are Hodag Nonionic™ 1108-F and Synperonic™ PE/F108. In one embodiment, the surface modifier comprises a combination of a polyoxyethylene sorbitan fatty acid ester and a phosphatidyl glycerol salt (in particular egg phosphatidyl glycerol sodium).

Preferably, the surface modifier is a poloxamer such as Pluronic™ F108 (poloxamer 338) or a polysorbate (Tween, e.g. Tween 20). In a particularly preferred embodiment, the surface modifier is a poloxamer such as Pluronic™ F108 (poloxamer 338). In another particularly preferred embodiment, the surface modifier is a polysorbate (Tween).

In an embodiment, the relative amount (w/w) of the drug to the surface modifier is from about 1:2 to about 20:1, preferably from about 1:1 to about 20:1, or from about 1:1 to about 10:1, e.g. about 4:1 to about 6:1.

In an embodiment, the micro- or nanoparticles of the invention comprise a drug as defined herein and one or more surface modifiers as defined herein wherein the amount of drug is at least about 50% by weight of the micro- or nanoparticles, at least about 80% by weight of the micro- or nanoparticles, at least about 85% by weight of the micro- or nanoparticles, at least about 90% by weight of the micro- or nanoparticles, at least about 95% by weight of the micro- or nanoparticles, or at least about 99% by weight of the micro- or nanoparticles.

In an embodiment, the suspension comprises a pharmaceutically acceptable aqueous carrier in which the drug micro- or nanoparticles are suspended. The pharmaceutically acceptable aqueous carrier comprises sterile water, e.g. water for injection, optionally in admixture with other pharmaceutically acceptable ingredients. The latter comprise any ingredients for use in injectable formulations. These ingredients may be selected from one or more of a suspending agent, a buffering agent, a pH adjusting agent, a preservative, an isotonizing agent, a surface modifier, a chelating agent and the like ingredients. In one embodiment, said ingredients are selected from one or more of a suspending agent, a buffering agent, a pH adjusting agent, and optionally, a preservative and an isotonizing agent. Particular ingredients may function as two or more of these agents simultaneously, e.g. behave like a preservative and a buffering agent, or behave like a buffering agent and an isotonizing agent. In an embodiment said ingredients are selected from one or more of a buffering agent, a pH adjusting agent, an isotonizing agent, a chelating agent and a surface modifier. In an embodiment said ingredients are selected from one or more of a buffering agent, a pH adjusting agent, an isotonizing agent, and a chelating agent.

In an embodiment, the suspension additionally comprises a buffering agent and/or a pH adjusting agent. Suitable buffering agents and pH adjusting agents should be used in amount sufficient to generate a pH of from about 3.5 to about 9, preferably to generate a pH of from about pH 6.5 to about pH 9), more preferably to generate a pH range of from about 6.5 to about 7.5. Particular buffering agents are the salts of weak acids. Buffering and pH adjusting agents that can be added may be selected from tartaric acid, maleic acid, glycine, sodium lactate/lactic acid, ascorbic acid, sodium citrates/citric acid, sodium acetate/acetic acid, sodium bicarbonate/carbonic acid, sodium succinate/succinic acid, sodium benzoate/benzoic acid, sodium phosphates, tris(hydroxymethyl)aminomethane, sodium bicarbonate/sodium carbonate, ammonium hydroxide, benzene sulfonic acid, benzoate sodium/acid, diethanolamine, glucono delta lactone, hydrochloric acid, hydrogen bromide, lysine, methanesulfonic acid, monoethanolamine, sodium hydroxide, tromethamine, gluconic, glyceric, gluratic, glutamic, ethylene diamine tetraacetic (EDTA), triethanolamine, including mixtures thereof. In an embodiment, the buffering agent is a sodium phosphate buffer, e.g. sodium dihydrogen phosphate monohydrate. In an embodiment the pH adjusting agent is sodium hydroxide.

In an embodiment, the suspension additionally comprises a preservative. Preservatives comprise antimicrobials and anti-oxidants which can be selected from the group consisting of benzoic acid, benzyl alcohol, butylated hydroxyanisole (BHA), butylated hydroxytoluene (BHT), chlorbutol, a gallate, a hydroxybenzoate, EDTA, phenol, chlorocresol, metacresol, benzethonium chloride, myristyl-γ-piccolinium chloride, phenylmercuric acetate and thimerosal. Radical scavengers include BHA, BHT, Vitamin E and ascorbyl palmitate, and mixtures thereof. Oxygen scavengers include sodium ascorbate, sodium sulfite, L-cysteine, acetylcysteine, methionine, thioglycerol, acetone sodium bisulfite, isoacorbic acid, hydroxypropyl cyclodextrin. Chelating agents include sodium citrate, sodium EDTA, citric acid and malic acid. In an embodiment, the chelating agent is citric acid, e.g. citric acid monohydrate.

In an embodiment, the suspension additionally comprises an isotonizing agent. An isotonizing agent or isotonifier may be present to ensure isotonicity of the pharmaceutical compositions of the present invention, and includes sugars such as glucose, dextrose, sucrose, fructose, trehalose, lactose; polyhydric sugar alcohols, preferably trihydric or higher sugar alcohols, such as glycerin, erythritol, arabitol, xylitol, sorbitol and mannitol.

Alternatively, sodium chloride, sodium sulfate, or other appropriate inorganic salts may be used to render the solutions isotonic. These isotonifiers can be used alone or in combination. The suspensions conveniently comprise from 0 to 10% (w/v), in particular 0 to 6% (w/v) of isotonizing agent. Of interest are nonionic isotonifiers, e.g. glucose, mannitol, as electrolytes may affect colloidal stability.

In an embodiment, each administration comprises up to about 600 mL of the suspension described herein, i.e. the volume of the suspension comprising the drug in the form of micro- or nanoparticles may have a volume of up to 600 mL. In an embodiment, each administration comprises up to about 300 mL of the suspension. In another embodiment, each administration comprises up to about 200 mL of the suspension. In another embodiment, each administration comprises up to about 150 mL of the suspension. In another embodiment, each administration comprises up to about 25 mL of the suspension.

In an embodiment, each administration comprises at least about 5 mL of the suspension. In an embodiment, each administration comprises at least about 10 mL of the suspension. In a preferred embodiment, each administration comprises at least about 25 mL of the suspension.

In an embodiment, each administration comprises from about 5 mL to about 600 mL of the suspension. In a preferred embodiment, each administration comprises from about 25 mL to about 600 mL of the suspension. In another preferred embodiment, each administration comprises from about 25 mL to about 300 mL of the suspension. In another preferred embodiment, each administration comprises from about 25 mL to about 200 mL of the suspension. In another preferred embodiment, each administration comprises from about 5 mL to about 150 mL of the suspension. In another preferred embodiment, each administration comprises from about 5 mL to about 25 mL of the suspension.

In an embodiment, the drug (which is in the form of micro- or nanoparticles in suspension) is provided in a separate pharmaceutical composition from the hyaluronidase. As discussed further herein (e.g. in the section titled “Use of the drug and hyaluronidase in the invention”), the separate pharmaceutical composition may be administered sequentially with a pharmaceutical composition comprising the hyaluronidase of the invention, or the separate pharmaceutical composition may be admixed with a pharmaceutical composition comprising the hyaluronidase of the invention prior to administration of the resulting admixed pharmaceutical composition.

In another embodiment, the drug (which is in the form of micro- or nanoparticles in suspension) is provided in the same pharmaceutical composition as the hyaluronidase, i.e. the drug is formulated in a combined pharmaceutical composition with the hyaluronidase.

Each administration of the drug may comprise the drug in an amount of from about 25 mg per millilitre suspension (mg/mL) to about 400 mg/mL, preferably from about 100 mg/mL to about 350 mg/mL, more preferably from about 200 mg/mL to about 300 mg/mL, or from about 20 mg/mL to about 50 mg/mL. Thus, the amount of the drug in the pharmaceutical composition, i.e. the separate or combined pharmaceutical composition defined herein, per millilitre of suspension may be from about 25 mg to about 400 mg, preferably from about 100 mg to about 350 mg, more preferably from about 200 mg to about 300 mg, or from about 20 mg/mL to about 50 mg/mL.

In an embodiment, the dose to be administered may be calculated on a basis of about 300 mg to about 70 g/month. In another embodiment, the dose to be administered may be calculated on a basis of about 1 g to about 50 g/month. In another embodiment, the dose to be administered may be calculated on a basis of about 10 g to about 50 g/month. In another embodiment, the dose to be administered may be calculated on a basis of about 5 g to about 20 g/month. In another embodiment, the dose to be administered may be calculated on a basis of about 1 g to about 5 g/month Doses for other dosing regimens can readily be calculated by multiplying the monthly dose with the number of months between each administration. For example, in case of a dose of 1 g/month, and in case of a time interval of 6 months between each administration, the dose to be administered in each administration is 6 g.

In an embodiment, the drug is rilpivirine or a pharmaceutically acceptable salt thereof and for the treatment of a disease or disorder, e.g. HIV infection, the dose to be administered may be calculated on a basis of about 300 mg to about 1200 mg/month, or about 450 mg to about 1200 mg/month, or about 450 mg to about 900 mg/month, or about 600 mg to about 900 mg/month, or about 450 mg to about 750 mg/month, or 450 mg/month, or 600 mg/month, or 750 mg/month, or 900 mg/month. Doses for other dosing regimens can readily be calculated by multiplying the monthly dose with the number of months between each administration. For example, in case of a dose of 450 mg/month, and in case of a time interval of 6 months between each administration, the dose to be administered in each administration is 2700 mg. The indicated “mg” corresponds to mg of rilpivirine (i.e. rilpivirine in its free base form). Thus, by way of example, 1 mg of rilpivirine (i.e. rilpivirine in its free base form) corresponds to 1.1 mg of rilpivirine hydrochloride.

In an embodiment, the drug is rilpivirine or a pharmaceutically acceptable salt thereof and for the treatment of a disease or disorder, e.g. HIV infection, the dose to be administered may be calculated on a basis of about 300 mg to about 1200 mg/4 weeks (28 days), or about 450 mg to about 1200 mg/4 weeks (28 days), or about 450 mg to about 900 mg/4 weeks (28 days), or about 600 mg to about 900 mg/4 weeks (28 days), or about 450 mg to about 750 mg/4 weeks (28 days) or 450 mg/4 weeks (28 days), or 600 mg/4 weeks (28 days), or 750 mg/4 weeks (28 days) or 900 mg/4 weeks (28 days). Doses for other dosing regimens can readily be calculated by multiplying the week or day dose with the number of weeks between each administration. For example, in case of a dose of 450 mg/4 weeks (28 days), and in case of a time interval of 24 weeks between each administration, the dose to be administered in each administration is 2700 mg. Or for example, in case of a dose of 750 mg/4 weeks (28 days), and in case of a time interval of 24 weeks between each administration, the dose to be administered in each administration is 4500 mg. The indicated “mg” corresponds to mg of rilpivirine (i.e. rilpivirine in its free base form). Thus, by way of example, 1 mg of rilpivirine (i.e. rilpivirine in its free base form) corresponds to 1.1 mg of rilpivirine hydrochloride.

In an embodiment, for the treatment of HIV infection, each administration of rilpivirine or a pharmaceutically acceptable salt thereof may comprise from about 900 mg to about 28800 mg (e.g. from about 900 mg to about 14400 mg, or from about 900 mg to about 7200 mg, or from about 900 mg to about 3600 mg), preferably from about 1200 mg to about 14400 mg, preferably from about 1350 mg to about 13200 mg, preferably from about 1500 mg to about 12000 mg, (e.g. from about 3000 mg to about 12000 mg), preferably from about 1800 mg to about 10800 mg (e.g. from about 2700 mg to about 10800 mg, or from about 1800 mg to about 3600 mg), most preferably from about 1800 mg to about 7200 mg or from about 2700 mg to about 4500 mg of the rilpivirine or pharmaceutically acceptable salt thereof.

Thus, the amount of the rilpivirine or pharmaceutically acceptable salt thereof in the pharmaceutical composition, i.e. the separate or combined pharmaceutical composition defined herein, may be from about 900 mg to about 28800 mg (e.g. from about 900 mg to about 14400 mg, or from about 900 mg to about 7200 mg, or from about 900 mg to about 3600 mg), preferably from about 1200 mg to about 14400 mg, preferably from about 1350 mg to about 13200 mg, preferably from about 1500 mg to about 12000 mg, (e.g. from about 3000 mg to about 12000 mg), preferably from about 1800 mg to about 10800 mg (e.g. from about 2700 mg to about 10800 mg, or from about 1800 mg to about 3600 mg), most preferably from about 1800 mg to about 7200 mg or from about 2700 mg to about 4500 mg. The indicated “mg” corresponds to mg of rilpivirine (i.e. rilpivirine in its free base form). Thus, by way of example, 1 mg of rilpivirine (i.e. rilpivirine in its free base form) corresponds to 1.1 mg of rilpivirine hydrochloride.

In a preferred embodiment the drug is rilpivirine and the dose of rilpivirine is from about 900 mg to about 2700 mg.

In an embodiment, the drug is rilpivirine or a pharmaceutically acceptable salt thereof and, in the instance of prevention of HIV infection, each administration of rilpivirine or pharmaceutically acceptable salt thereof may comprise the same dosing as for therapeutic applications as described above.

In an embodiment, the drug is rilpivirine or a pharmaceutically acceptable salt thereof, and the drug in the pharmaceutical composition, i.e. the separate or combined pharmaceutical composition defined herein, is used in an amount such that the blood plasma concentration of the drug in the subject is kept at a level above about 12 ng/ml, preferably ranging from about 12 ng/ml to about 100 ng/ml, preferably about 12 ng/ml to about 500 ng/ml, for at least three months after administration, or at least 6 months after administration, or at least 9 months after administration, or at least 1 year after administration, or at least 2 years after each administration. In a preferred embodiment, the drug in the pharmaceutical composition is used in an amount such that the blood plasma concentration of the drug in the subject is kept at a level of from 12 ng/ml to 100 ng/ml for at least 6 months.

In a particular embodiment, the drug is formulated and administered as micro- or nanoparticles in suspension wherein the formulation comprises the following components:
a drug as defined herein, in particular rilpivirine;
a surface modifier as defined herein, in particular poloxamer 338;
an isotonizing agent as defined herein, in particular glucose monohydrate;
a buffering agent as defined herein, in particular sodium dihydrogen phosphate;
a chelating agent as defined herein, in particular citric acid monohydrate;
a pH adjusting agent as defined herein, in particular sodium hydroxide; and water, in particular water for injection.

In another particular embodiment, the drug is rilpivirine which is formulated and administered as micro- or nanoparticles in suspension wherein the formulation comprises the following components:
rilpivirine or a pharmaceutically acceptable salt thereof, in particular rilpivirine;
poloxamer 338;
glucose monohydrate;
sodium dihydrogen phosphate;
citric acid monohydrate;
sodium hydroxide; and
water, in particular water for injection.

In one embodiment, the aqueous suspensions may comprise by weight, based on the total volume of the suspension:
(a) from 3% to 50% (w/v), or from 10% to 40% (w/v), or from 10% to 30% (w/v), of drug, in particular rilpivirine or a pharmaceutically acceptable salt thereof; in particular rilpivirine; or in particular apalutamide;
(b) from 0.5% to 10% (w/v), or from 0.5% to 5% (w/v), or from 0.5% to 2% (w/v) of a surface modifier; in particular poloxamer 338;
(c) from 0% to 10% (w/v), or from 0% to 5% (w/v), or from 0% to 2% (w/v), or from 0% to 1% (w/v) of one or more buffering agents; in particular sodium dihydrogen phosphate;
(d) from 0% to 10% (w/v), or from 0% to 6% (w/v), or from 0% to 5% (w/v), or from 0% to 3% (w/v), or from 0% to 2% (w/v) of an isotonizing agent; in particular glucose monohydrate;
(e) from 0% to 2% (w/v), or from 0% to 1% (w/v), or from 0% to 0.5% (w/v), or from 0% to 0.1% (w/v) of a pH adjusting agent; in particular sodium hydroxide;
(f) from 0% to 2% (w/v), or from 0% to 1% (w/v), or from 0% to 0.5% (w/v), or from 0% to 0.1% (w/v) of a chelating agent; in particular citric acid monohydrate;
(g) from 0% to 2% (w/v) preservatives; and
(h) water for injection q.s. ad 100%.

In one embodiment, the aqueous suspensions may comprise by weight, based on the total volume of the suspension:
(a) from 3% to 50% (w/v), or from 10% to 40% (w/v), or from 10% to 30% (w/v), of drug, in particular rilpivirine or a pharmaceutically acceptable salt thereof; in particular rilpivirine, or in particular apalutamide;
(b) from 0.5% to 10% (w/v), or from 0.5% to 5% (w/v), or from 0.5% to 2% (w/v) of a surface modifier; in particular poloxamer 338:
(c) from 0% to 10% (w/v), or from 0% to 5% (w/v), or from 0% to 2% (w/v), or from 0% to 1% (w/v) of one or more buffering agents; in particular sodium dihydrogen phosphate;
(d) from 0% to 10% (w/v), or from 0% to 6% (w/v), or from 0% to 5% (w/v), or from 0% to 3% (w/v), or from 0% to 2% (w/v) of an isotonizing agent; in particular glucose monohydrate;
(e) from 0% to 2% (w/v), or from 0% to 1% (w/v), or from 0% to 0.5% (w/v), or from 0% to 0.1% (w/v) of a pH adjusting agent; in particular sodium hydroxide;
(f) from 0% to 2% (w/v), or from 0% to 1% (w/v), or from 0% to 0.5% (w/v), or from 0% to 0.1% (w/v) of a chelating agent; in particular citric acid monohydrate; and
(g) water for injection q.s. ad 100%.

In a particular embodiment of the invention, the rilpivirine or pharmaceutically acceptable salt thereof is formulated (and administered) as a suspension of micro- or nanoparticles wherein the suspension comprises the following components in the following amounts:
(a) Rilpivirine (300 mg);
(b) Poloxamer 338 (50 mg); and
(c) Water for injection (ad 1 ml).

Alternatively, these components may be used in different amounts but with the same weight ratio between components and the total volume (made up by water for injection) scaled by the same value.

In a particular embodiment, the rilpivirine or pharmaceutically acceptable salt thereof is formulated (and administered) as a suspension of micro- or nanoparticles wherein the suspension comprises the following components in the following amounts:
(a) Rilpivirine (300 mg);
(b) Poloxamer 338 (50 mg);
(c) Glucose monohydrate (19.25 mg);
(d) Sodium dihydrogen phosphate (2.00 mg);
(e) Citric acid monohydrate (1.00 mg);
(f) Sodium Hydroxide (0.866 mg); and
(g) Water for injection (ad 1 ml).

Alternatively, these components may be used in different amounts but with the same weight ratio between components and the total volume (made up by water for injection) scaled by the same value.

In an embodiment, the suspension of drug as described herein is administered by a manual injection process.

Hyaluronidase

Hyaluronidase is an enzyme that degrades hyaluronic acid (HA) and lowers the viscosity of hyaluronan in the extracellular matrix. Because of this property, it can be used to increase dispersion and absorption of injected active pharmaceutical ingredients. Enzymatic activity of hyaluronidase, including rHuPH20, can be defined by units per mL (U/mL) or by total enzyme activity in a particular formulation (U).

It is generally known that the delivery of hyaluronidases (E.C. 3.2.1.35/36) into the tissue improves the penetration of drugs. Administration of hyaluronidase thus represents a method of increasing the dispersion and improving the absorption of drugs.

The term “hyaluronidase” as used herein means any enzyme that degrades hyaluronic acid and lowers the viscosity of hyaluronan in the extracellular matrix. For the avoidance of doubt, the hyaluronidase described herein is not the drug described herein.

In a preferred embodiment, the hyaluronidase is recombinant hyaluronidase. In a particularly preferred embodiment, the hyaluronidase is recombinant human hyaluronidase, e.g. rHuPH20. In an embodiment of the invention, rHuPH20 is defined by the amino acid sequence available under CAS Registry No. 757971-58-7. Further information regarding rHuPH20 is provided in Int. Pat. Publ. No. WO2004/078140. In an embodiment of the invention, the amino acid sequence of rHuPH20 comprises SEQ ID NO: 1. In some embodiments of the invention, the hyaluronidase is a variant of rHuPH20 having an amino acid sequence of rHuPH20 that comprises SEQ ID NO: 2, namely residues 36-482 of wild type human hyaluronidase. In some embodiments of the invention, the hyaluronidase is a variant of rHuPH20 having an amino acid sequence that comprises SEQ ID NO: 3. In some embodiments of the invention, the hyaluronidase is a variant of rHuPH20 having an amino acid sequence that comprises SEQ ID NO: 4. In some embodiments of the invention, the hyaluronidase is a variant of rHuPH20 having an amino acid sequence that comprises SEQ ID NO: 5.

| | |
|---|---|
| SEQ ID NO: 1: | LNFRAPPVIPNVPFLWAWNAPSEFCLGKFDEPLDMSLFSFIGSPRIN |
| rHuPH20 | ATGQGVTIFYVDRLGYYPYIDSITGVTVNGGIPQKISLQDHLDKAK |
| | KDITFYMPVDNLGMAVIDWEEWRPTWARNWKPKDVYKNRSIEL |
| | VQQQNVQLSLTEATEKAKQEFEKAGKDFLVETIKLGKLLRPNHLW |
| | GYYLFPDCYNHHYKKPGYNGSCFNVEIKRNDDLSWLWNESTALY |
| | PSIYLNTQQSPVAATLYVRNRVREAIRVSKIPDAKSPLPVFAYTRIV |
| | FTDQVLKFLSQDELVYTFGETVALGASGIVIWGTLSIMRSMKSCLL |
| | LDNYMETILNPYIINVTLAAKMCSQVLCQEQGVCIRKNWNSSDYL |
| | HLNPDNFAIQLEKGGKFTVRGKPTLEDLEQFSEKFYCSCYSTLSCK |
| | EKADVKDTDAVDVCIADGVCIDAFLKPPMETEEP |
| SEQ ID NO: 2: | LNFRAPPVIPNVPFLWAWNAPSEFCLGKFDEPLDMSLFSFIGSPRIN |
| rHuPH20 variant | ATGQGVTIFYVDRLGYYPYIDSITGVTVNGGIPQKISLQDHLDKAK |
| 1 | KDITFYMPVDNLGMAVIDWEEWRPTWARNWKPKDVYKNRSIEL |
| | VQQQNVQLSLTEATEKAKQEFEKAGKDFLVETIKLGKLLRPNHLW |
| | GYYLFPDCYNHHYKKPGYNGSCFNVEIKRNDDLSWLWNESTALY |
| | PSIYLNTQQSPVAATLYVRNRVREAIRVSKIPDAKSPLPVFAYTRIV |
| | FTDQVLKFLSQDELVYTFGETVALGASGIVIWGTLSIMRSMKSCLL |
| | LDNYMETILNPYIINVTLAAKMCSQVLCQEQGVCIRKNWNSSDYL |
| | HLNPDNFAIQLEKGGKFTVRGKPTLEDLEQFSEKFYCSCYSTLSCK |
| | EKADVKDTDAVDVCIADGVCIDAFLKPPMETEEPQIFY |
| SEQ ID NO: 3: | LNFRAPPVIPNVPFLWAWNAPSEFCLGKFDEPLDMSLFSFIGSPRIN |
| rHuPH20 variant | ATGQGVTIFYVDRLGYYPYIDSITGVTVNGGIPQKISLQDHLDKAK |
| 2 | KDITFYMPVDNLGMAVIDWEEWRPTWARNWKPKDVYKNRSIEL |
| | VQQQNVQLSLTEATEKAKQEFEKAGKDFLVETIKLGKLLRPNHLW |
| | GYYLFPDCYNHHYKKPGYNGSCFNVEIKRNDDLSWLWNESTALY |
| | PSIYLNTQQSPVAATLYVRNRVREAIRVSKIPDAKSPLPVFAYTRIV |
| | FTDQVLKFLSQDELVYTFGETVALGASGIVIWGTLSIMRSMKSCLL |
| | LDNYMETILNPYIINVTLAAKMCSQVLCQEQGVCIRKNWNSSDYL |
| | HLNPDNFAIQLEKGGKFTVRGKPTLEDLEQFSEKFYCSCYSTLSCK |
| | EKADVKDTDAVDVCIADGVCIDAFLKPPMETEEPQIF |
| SEQ ID NO: 4: | LNFRAPPVIPNVPFLWAWNAPSEFCLGKFDEPLDMSLFSFIGSPRIN |
| rHuPH20 variant | ATGQGVTIFYVDRLGYYPYIDSITGVTVNGGIPQKISLQDHLDKAK |
| 3 | KDITFYMPVDNLGMAVIDWEEWRPTWARNWKPKDVYKNRSIEL |
| | VQQQNVQLSLTEATEKAKQEFEKAGKDFLVETIKLGKLLRPNHLW |
| | GYYLFPDCYNHHYKKPGYNGSCFNVEIKRNDDLSWLWNESTALY |
| | PSIYLNTQQSPVAATLYVRNRVREAIRVSKIPDAKSPLPVFAYTRIV |
| | FTDQVLKFLSQDELVYTFGETVALGASGIVIWGTLSIMRSMKSCLL |
| | LDNYMETILNPYIINVTLAAKMCSQVLCQEQGVCIRKNWNSSDYL |
| | HLNPDNFAIQLEKGGKFTVRGKPTLEDLEQFSEKFYCSCYSTLSCK |
| | EKADVKDTDAVDVCIADGVCIDAFLKPPMETEEPQI |
| SEQ ID NO: 5: | LNFRAPPVIPNVPFLWAWNAPSEFCLGKFDEPLDMSLFSFIGSPRIN |
| rHuPH20 variant | ATGQGVTIFYVDRLGYYPYIDSITGVTVNGGIPQKISLQDHLDKAK |
| 4 | KDITFYMPVDNLGMAVIDWEEWRPTWARNWKPKDVYKNRSIEL |
| | VQQQNVQLSLTEATEKAKQEFEKAGKDFLVETIKLGKLLRPNHLW |
| | GYYLFPDCYNHHYKKPGYNGSCFNVEIKRNDDLSWLWNESTALY |
| | PSIYLNTQQSPVAATLYVRNRVREAIRVSKIPDAKSPLPVFAYTRIV |
| | FTDQVLKFLSQDELVYTFGETVALGASGIVIWGTLSIMRSMKSCLL |
| | LDNYMETILNPYIINVTLAAKMCSQVLCQEQGVCIRKNWNSSDYL |
| | HLNPDNFAIQLEKGGKFTVRGKPTLEDLEQFSEKFYCSCYSTLSCK |
| | EKADVKDTDAVDVCIADGVCIDAFLKPPMETEEPQ |

In an embodiment, the hyaluronidase of the invention is formulated in a separate pharmaceutical composition. As discussed further herein (e.g. in the section titled “Use of the drug and hyaluronidase in the invention”), the separate pharmaceutical composition may be administered sequentially with a pharmaceutical composition comprising the drug, or the separate pharmaceutical composition may be admixed extemporaneously with a pharmaceutical composition comprising the drug prior to administration of the resulting admixed pharmaceutical composition.

In another embodiment, the hyaluronidase of the invention is formulated in the same pharmaceutical composition as the drug, i.e. the hyaluronidase is formulated as a combined pharmaceutical composition (with the drug).

In an embodiment, the hyaluronidase is in the form of a solution, preferably wherein the concentration of the hyaluronidase in the solution is from about 50 to about 20,000 U/mL, preferably about 50 to about 10,000 U/mL, from about 50 to about 5000 U/mL, from about 500 to about 2000 U/mL. In an embodiment of the invention, the hyaluronidase is in the form of a solution, preferably wherein the concentration of the hyaluronidase in the solution is about 500 U/mL. In an embodiment of the invention, the hyaluronidase is in the form of a solution, preferably wherein the concentration of the hyaluronidase in the solution is about 750 U/mL. In an embodiment of the invention, the hyaluronidase is in the form of a solution, preferably wherein the concentration of the hyaluronidase in the solution is about 1000 U/mL. In an embodiment of the invention, the hyaluronidase is in the form of a solution, preferably wherein the concentration of the hyaluronidase in the solution is about 1250 U/mL. In an embodiment of the invention, the hyaluronidase is in the form of a solution, preferably wherein the concentration of the hyaluronidase in the solution is about 1500 U/mL. In an embodiment of the invention, the hyaluronidase is in the form of a solution, preferably wherein the concentration of the hyaluronidase in the solution is about 1750 U/mL. In an embodiment of the invention, the hyaluronidase is in the form of a solution, preferably wherein the concentration of the hyaluronidase in the solution is about 2000 U/mL.

In some embodiments, the hyaluronidase containing composition comprises hyaluronidase at a dose of about 1,000 U, 2,000 U, 3,000 U, 4,000 U, about 5,000 U, about 6,000 U, about 7,000 U, about 8,000 U, about 9,000 U, about 10,000 U, about 11,000 U, about 12,000 U, about 13,000 U, about 14,000 U, about 15,000 U, about 16,000 U, about 17,000 U, about 18,000 U, about 19,000 U, about 20,000 U, about 21,000 U, about 22,000 U, about 23,000 U, about 24,000 U, about 25,000 U, about 26,000 U, about 27,000 U, about 30,000 U, about 31,000 U, about 32,000 U, about 33,000 U, about 34,000 U, about 35,000 U, about 36,000 U, about 37,000 U, about 38,000 U, about 39,000 U, about 40,000 U, or any value in between. In some embodiments, where the hyaluronidase is administered sequentially with a pharmaceutical composition comprising the drug, the hyaluronidase containing composition comprises hyaluronidase at a dose of about 1,000 U, 2,000 U, 3,000 U, 4,000 U, about 5,000 U, about 6,000 U, about 7,000 U, about 8,000 U, about 9,000 U, about 10,000 U, or any value in between. In a preferred embodiment the hyaluronidase containing composition comprises hyaluronidase at a dose of about 2,000 U. In some embodiments, where the hyaluronidase is admixed extemporaneously with a pharmaceutical composition comprising the drug prior to administration of the resulting admixed pharmaceutical composition, the admixed composition comprises hyaluronidase at a dose of about 11,000 U, about 12,000 U, about 13,000 U, about 14,000 U, about 15,000 U, about 16,000 U, about 17,000 U, about 18,000 U, about 19,000 U, about 20,000 U, about 21,000 U, about 22,000 U, about 23,000 U, about 24,000 U, about 25,000 U, about 26,000 U, about 27,000 U, about 30,000 U, about 31,000 U, about 32,000 U, about 33,000 U, about 34,000 U, about 35,000 U, about 36,000 U, about 37,000 U, about 38,000 U, about 39,000 U, about 40,000 U, or any value in between.

In a particular embodiment, the hyaluronidase is formulated as a solution in a separate pharmaceutical composition, i.e. as a solution without the drug, and the separate pharmaceutical composition comprises the following components:
from about 50 U/mL to about 10,000 U/mL rHuPH20;
from about 5 mM to about 50 mM histidine;
from about 50 mM to about 400 mM sorbitol;
from about 0.1 mg/mL to about 2.5 mg/mL methionine; and
from about 0.01% (w/v) to about 0.1% (w/v) polysorbate 20 buffer.

Use of the Drug and Hyaluronidase in the Invention

In a first aspect there is provided a method for the treatment or prevention of a disease or disorder in a subject in need thereof, the method comprising administering to the subject a drug effective in the treatment or prevention of the disease or disorder in the form of micro- or nanoparticles in suspension by intramuscular injection or subcutaneous injection, wherein the drug is administered in combination with a hyaluronidase that is administered by intramuscular injection or subcutaneous injection, and wherein the drugand the hyaluronidase are administered intermittently at a time interval of about three months to about two years.

Thus, the method for treatment or prevention described herein involves administering a drug and a hyaluronidase multiple times, and the time interval between an administration of the drug and the hyaluronidase and a subsequent administration of the drug and the hyaluronidase is about three months to about two years, i.e. the drug and hyaluronidase according to the invention is administered to a subject as described herein, and then after a period of from three months to two years the drug and hyaluronidase according to the invention is administered again to the subject as defined herein.

The terms “is administered” and “are administered” as used herein in relation to the methods for treatment or prevention and uses described herein may encompass the terms “is to be administered” and “are to be administered”, respectively.

In a preferred embodiment, the subject is a human.

The drug and the hyaluronidase may be administered simultaneously or sequentially. In an embodiment, the drug and the hyaluronidase are administered sequentially, i.e. one after the other, preferably within 24 hours of each other, preferably within 1 hour of each other, preferably within 30 minutes of each other, preferably within 10 minutes of each other, more preferably within 5 minutes of each other. Preferably, the hyaluronidase is administered before administration of the drug. In another embodiment, the drug and the hyaluronidase are administered simultaneously.

When the drug and the hyaluronidase are administered sequentially, they are formulated in separate pharmaceutical compositions. These separate pharmaceutical compositions are described further in the sections titled “Drug” and “Hyaluronidase” herein.

When the drug and the hyaluronidase are administered sequentially, they are both administered by the same method, i.e. subcutaneous or intramuscular injection. Further, they are both administered at the same site. By same site it is meant that the injection sites are within 15 cm of each other, within 12 cm of each other, or within 8 cm of each other. Preferably the injection sites are within 10 cm of each other, more preferably within 5 cm of each other, even more preferably within 1 cm of each other. This allows the hyaluronidase to exert its effect in increasing the tolerability of the injection of drug, in particular rilpivirine or pharmaceutically acceptable salt thereof or apalutamide.

When the drug and hyaluronidase are administered simultaneously, they may both be administered at the same site, i.e. simultaneously via the same needle. When the drug and hyaluronidase are administered simultaneously, the drug and hyaluronidase may be provided in combined pharmaceutical composition, i.e. a pharmaceutical composition comprising both the drug and the hyaluronidase. This combined pharmaceutical composition is described further in the sections titled “The drug(s) used in the invention” and “Hyaluronidase” herein. When the drug and hyaluronidase are administered simultaneously, the drug and hyaluronidase may also be provided as separate pharmaceutical compositions which are admixed (i.e. to provide an admixed pharmaceutical formulation) extemporaneously prior to administration.

The combined pharmaceutical composition of the invention is surprisingly stable on storage, i.e. the hyaluronidase is active even after being combined with the drug, extemporaneously prior to administration, e.g. for at least 4 hours at room temperature, or for 24 hours or longer, in particular when stored at 2-8° C.

In an embodiment, the drug and the hyaluronidase are administered at the same injection site sequentially, through the same needle that has not been removed from the injection site, e.g. the skin.

The drug and hyaluronidase of the invention are administered such that the time interval between administrations (i.e. the dosing interval) is about three months to about two years. That is, the drug is administered (e.g. simultaneously or sequentially) with the hyaluronidase and then following a time interval of about three months to about two years the drug is administered (e.g. simultaneously or sequentially) with the hyaluronidase again.

It has been found that the extended, sustained or prolonged release of drugs when administered in the form of micro- or nanoparticles in suspension by intramuscular or subcutaneous injection can be maintained when administering the drug with a hyaluronidase as defined herein. This surprising effect is discussed in detail in Examples 1 and 2.

In an embodiment, the time interval described herein is about three months to about 1.5 years. In an embodiment, the time interval described herein is about two years. In a preferred embodiment, the time interval described herein is about three months to about one year. In another preferred embodiment, the time interval described herein is about three months to about six months. In another preferred embodiment, the time interval described herein is about six months to about 1 year. In another preferred embodiment, the time interval described herein is about three months. In another preferred embodiment, the time interval described herein is about six months. In another preferred embodiment, the time interval described herein is about 1 year.

The drug and the hyaluronidase are administered by subcutaneous injection or intramuscular injection. Preferably, the drug and hyaluronidase are administered by subcutaneous injection (either in the same combined pharmaceutical composition or in separate pharmaceutical compositions).

The drug and the hyaluronidase of the invention are used in a method for the treatment or prevention of a disease or disorder in a subject in need thereof, i.e. they are for use in therapy. The drug is administered in a therapeutically effective amount. By “therapeutically effective amount” it is meant an amount sufficient to provide a therapeutic effect.

In a particular embodiment, the drug and the hyaluronidase are used in a method for the treatment of a disease of disorder, in particular for the treatment of HIV infection or for the treatment of cancer, in a subject in need thereof as described herein, wherein the suspension comprises a pharmaceutically acceptable aqueous carrier in which the drug is suspended in the form of micro- or nanoparticles, and the drug and the hyaluronidase are administered by subcutaneous injection, and preferably wherein a surface modifier, e.g. poloxamer 338 or Tween 20, is adsorbed to the surface of the micro- or nanoparticles.

In an embodiment, the drug and hyaluronidase of the invention are for use in a method for the treatment or prevention of chronic and long-term diseases and disorders, for example for treatment of chronic viral infection (such as chronic infection with Varicella-zoster virus, measles virus, HIV, hepatitis B virus, hepatitis C virus, hepatitis D virus or human cytomegalovirus), cancer, psychiatric diseases and disorders, mood disorders (such as bipolar, cyclothymic or depression), diabetes, hypertension, abnormal cholesterol and triglyceride levels, inflammatory disorders (such as allergy, asthma, autoimmune diseases, coeliac disease, hepatitis, inflammatory bowel disease, Crohn disease, gout, myositis, scleroderma, rheumatoid arthritis, lupus vasculitis, ankylosing spondylitis or chronic obstructive pulmonary disease), cystic fibrosis, multiple sclerosis, autoimmune disorders, neurodegenerative disorders (such as Parkinson Disease or Alzheimer disease), chronic pain, inherited metabolic disorders or epilepsy.

In an embodiment, the drug and hyaluronidase of the invention are for use in the treatment or prevention of HIV infection in a subject, i.e. an embodiment described herein relates to a method for treating or preventing HIV infection in a subject using the drug and hyaluronidase as defined herein. In an embodiment, the HIV is HIV type 1 (HIV-1). When the disease or disorder is HIV infection, the drug is preferably rilpivirine or a pharmaceutically acceptable salt thereof, more preferably rilpivirine,

As used herein the term “treatment of HIV infection” relates to the treatment of a subject infected with HIV. The term “treatment of HIV infection” also relates to the treatment of diseases associated with HIV infection, for example AIDS, or other conditions associated with HIV infection including thrombocytopaenia, Kaposi's sarcoma and infection of the central nervous system characterized by progressive demyelination, resulting in dementia and symptoms such as, progressive dysarthria, ataxia and disorientation, and further conditions where HIV infection has also been associated with, such as peripheral neuropathy, progressive generalized lymphadenopathy (PGL), and AIDS-related complex (ARC).

As used herein the term “prevention of HIV infection” relates to the prevention or avoidance of a subject (who is not infected with HIV) becoming infected with HIV. The source of infection can be various, a material containing HIV, in particular a body fluid that contains HIV such as blood or semen, or another subject who is infected with HIV. Prevention of HIV infection relates to the prevention of the transmission of the virus from the material containing HIV or from the HIV infected individual to an uninfected person, or relates to the prevention of the virus from entering the body of an uninfected person. Transmission of the HIV virus can be by any known cause of HIV transfer such as by sexual transmission or by contact with blood of an infected subject, e.g. medical staff providing care to infected subjects. Transfer of HIV can also occur by contact with HIV infected blood, e.g. when handling blood samples or with blood transfusion. It can also be by contact with infected cells, e.g. when carrying out laboratory experiments with HIV infected cells.

The term “treatment of HIV infection” refers to a treatment by which the viral load of HIV (represented as the number of copies of viral RNA in a specified volume of serum) is reduced. The more effective the treatment, the lower the viral load. Preferably the viral load should be reduced to as low levels as possible, e.g. below about 200 copies/ml, in particular below about 100 copies/ml, more in particular below 50 copies/ml, if possible below the detection limit of the virus. Reductions of viral load of one, two or even three orders of magnitude (e.g. a reduction in the order of about 10 to about 10², or more, such as about 103) are an indication of the effectiveness of the treatment. Another parameter to measure effectiveness of HIV treatment is the CD4 count, which in normal adults ranges from 500 to 1500 cells per μl. Lowered CD4 counts are an indication of HIV infection and once below about 200 cells per μl, AIDS may develop. An increase of CD4 count, e.g. with about 50, 100, 200 or more cells per μl, is also an indication of the effectiveness of anti-HIV treatment. The CD4 count in particular should be increased to a level above about 200 cells per μl, or above about 350 cells per μl. Viral load or CD4 count, or both, can be used to diagnose the degree of HIV infection.

The term “treatment of HIV infection” and similar terms refer to that treatment that lowers the viral load, or increases CD4 count, or both, as described above. The term “prevention of HIV infection” and similar terms refer to that situation where there is a decrease in the relative number of newly infected subjects in a population in contact with a source of HIV infection such as a material containing HIV, or a HIV infected subject. Effective prevention can be measured, for example, by measuring in a mixed population of HIV infected and non-infected individuals, if there is a decrease of the relative number of newly infected individuals, when comparing non-infected individuals treated with a pharmaceutical composition of the invention, and non-treated non-infected individuals. This decrease can be measured by statistical analysis of the numbers of infected and non-infected individuals in a given population over time.

In a particular embodiment, the invention relates to a method for the treatment or prevention of HIV infection, preferably HIV type 1 (HIV-1) infection, in a subject in need thereof, the method comprising administering to the subject rilpivirine or a pharmaceutically acceptable salt thereof in the form of micro- or nanoparticles in suspension, as described herein, in combination with a hyaluronidase, particularly rHuPH20, as described herein, wherein the rilpivirine or pharmaceutically acceptable salt thereof and the hyaluronidase are administered to the subject by intramuscular or subcutaneous injection, preferably subcutaneous injection, and wherein the rilpivirine or pharmaceutically acceptable salt thereof and the hyaluronidase are administered intermittently at a time interval of about three months to about two years, preferably about three months to about six months.

In a second aspect there is provided a drug and a hyaluronidase for use in therapy, wherein the drug is in the form of micro- or nanoparticles in suspension, wherein the drug and hyaluronidase are administered by intramuscular injection or subcutaneous injection, and wherein the drug and hyaluronidase are administered intermittently at a time interval of about three months to about two years.

It will be understood that all of the embodiments described herein in relation to the first aspect, e.g. the embodiments relating to the drug in the invention, hyaluronidase in the invention, and the uses of the drug and hyaluronidase in the invention, apply equivalently, i.e. are also disclosed herein in relation to, this second aspect of the invention.

In a third aspect there is provided products containing a drug and a hyaluronidase as a combined preparation for simultaneous, separate or sequential use in therapy by intramuscular injection or subcutaneous injection, wherein the drug is in the form of micro- or nanoparticles in suspension, and wherein the drug and the hyaluronidase are administered intermittently at a time interval of about three months to about two years.

It will be understood that all of the embodiments described herein in relation to the first aspect, e.g. the embodiments relating to the drug in the invention, hyaluronidase in the invention, and the uses of the drug and hyaluronidase in the invention, apply equivalently, i.e. are also disclosed herein in relation to, this third aspect of the invention.

In a fourth aspect there is provided a kit of parts comprising a drug and a hyaluronidase for simultaneous or sequential use in therapy by intramuscular injection or subcutaneous injection, wherein the drug is in the form of micro- or nanoparticles in suspension, and wherein the drug and the hyaluronidase are administered intermittently at a time interval of about three months to about two years.

It will be understood that all of the embodiments described herein in relation to the first aspect, e.g. the embodiments relating to the drug in the invention, hyaluronidase in the invention, and the uses of the drug and hyaluronidase in the invention, apply equivalently, i.e. are also disclosed herein in relation to, this fourth aspect of the invention.

In a fifth aspect there is provided a drug in the form of micro- or nanoparticles in suspension for use in therapy by intramuscular injection or subcutaneous injection, wherein the drug is administered in combination with a hyaluronidase that is administered by intramuscular injection or subcutaneous injection, and wherein the drug and the hyaluronidase are administered intermittently at a time interval of about three months to about two years.

It will be understood that all of the embodiments described herein in relation to the first aspect, e.g. the embodiments relating to the drug in the invention, hyaluronidase in the invention, and the uses of the drug and hyaluronidase in the invention, apply equivalently, i.e. are also disclosed herein in relation to, this fifth aspect of the invention.

In a sixth aspect there is provided use of a drug for the manufacture of a medicament for use in the treatment of a disease or disorder in a subject, wherein the drug is in the form of micro- or nanoparticles in suspension and is administered in combination with a hyaluronidase, wherein the drug and the hyaluronidase are administered to the subject by intramuscular injection or subcutaneous injection, and wherein the drug and the hyaluronidase are administered intermittently at a time interval of about three months to about two years.

It will be understood that all of the embodiments described herein in relation to the first aspect, e.g. the embodiments relating to the drug in the invention, hyaluronidase in the invention, and the uses of the drug and hyaluronidase in the invention, apply equivalently, i.e. are also disclosed herein in relation to, this sixth aspect of the invention.

In a seventh aspect there is provided a combination comprising a drug, in particular a drug having a molecular weight (MW) of less than 1000 Da, in particular a drug not being a biologic and having a molecular weight (MW) of less than 1000 Da, and a hyaluronidase, wherein the drug is in the form of micro- or nanoparticles in suspension.

It will be understood that all of the embodiments described herein in relation to the first aspect, e.g. the embodiments relating to the drug in the invention and the hyaluronidase in the invention apply equivalently, i.e. are also disclosed herein in relation to, this seventh aspect of the invention.

In an eighth aspect there is provided a kit of parts comprising a drug, in particular a drug having a molecular weight (MW) of less than 1000 Da, in particular a drug not being a biologic and having a molecular weight (MW) of less than 1000 Da, and a hyaluronidase, wherein the drug is in the form of micro- or nanoparticles in suspension.

It will be understood that all of the embodiments described herein in relation to the first aspect, e.g. the embodiments relating to the drug in the invention and the hyaluronidase in the invention apply equivalently, i.e. are also disclosed herein in relation to, this eighth aspect of the invention.

### GENERAL DEFINITIONS

The term “comprising” encompasses “including” as well as “consisting”, e.g. a composition “comprising” X may consist exclusively of X or may include something additional, e.g. X+Y. The term “comprising” used herein also encompasses “consisting essentially of”, e.g. a composition “comprising” X may consist of X and any other components that do not materially affect the essential characteristics of the composition.

The term “about” in relation to a numerical value Y is optional and means, for example, Y±10%.

When a time interval is expressed as a specified number of months, it runs from a given numbered day of a given month to the same numbered day of the month that falls the specified number of months later. Where the same numbered day does not exist in the month that falls the specified number of months later, the time interval runs into the following month for the same number of days it would have run if the same numbered day would exist in the month that falls the specified number of months later.

When a time interval is expressed as a number of years, it runs from a given date of a given year to the same date in the year that falls the specified number of years later. Where the same date does not exist in the year that falls the specified number of years later, the time interval runs for the same number of days it would have run if the same numbered day would exist in the month that falls the specified number of months later. In other words, if the time interval starts on 29th February of a given year but ends in a year where there is no 29th February, the time period ends instead on 1st March in that year.

The term “about” in relation to such a definition means that the time interval may end on a date that is ±10% of the time interval.

In an embodiment, the time interval may start up to 7 days before or after the start of the time interval and end up to 7 days before or after the end of the time interval.

All references cited herein are incorporated by reference in their entirety.

The invention will now be described with reference to the following examples. For the avoidance of doubt, this example does not limit the scope of the invention. Modifications may be made whilst remaining within the scope and spirit of the invention.

### EXAMPLES

### Example 1—Administration of a Drug and a Hyaluronidase

This example compares the plasma kinetics after administration of a drug suspension with the plasma kinetics following sequential administration of first a hyaluronidase solution then a drug suspension.

Preparation of Drug and Hyaluronidase Compositions

(a) Suspension of Drug

A 3.380 mL fill of 300 mg/mL suspension of rilpivirine (Dᵥ50=˜200 nm) was prepared in 4R glass vials with the following excipients:
(a) Poloxamer 338 (50 mg/ml)
(b) Glucose monohydrate (19.25 mg/ml)
(c) Sodium dihydrogen phosphate monohydrate (2.00 mg/ml)
(d) Citric acid monohydrate (1.00 mg/ml)
(e) Sodium hydroxide (0.866 mg/ml)
(f) Water for injection (q.s ad 3 mL)

The suspension was prepared as follows:

A buffer solution was prepared by dissolving citric acid monohydrate, sodium dihydrogen phosphate monohydrate, sodium hydroxide and, glucose monohydrate in water for injection in a stainless steel vessel. Poloxamer 338 was added to the buffer solution and mixed until dissolved. A first fraction of the poloxamer 338 buffer solution was passed sequentially through a pre-filter and 2 serially-connected sterile filters into a sterilized stainless steel vessel. The sterile drug substance (micronized irradiated) was aseptically dispersed, via a charging isolator, into the sterile solution. The remaining fraction of poloxamer 338 buffer solution was passed sequentially through a pre-filter and 2 serially-connected sterile filters into the milling vessel to make up the suspension concentrate. During and after addition of the drug substance, the suspension concentrate was mixed to wet and disperse the drug substance.

Milling of the Suspension Concentrate

The suspension concentrate in the milling vessel was aseptically milled by circulating through a sterilized stainless-steel milling chamber, using sterilized zirconia beads as grinding media. During the milling process, the suspension circulated between the milling chamber and the milling vessel by means of a peristaltic pump until the target particle size was achieved.

Dilution of the Suspension Concentrate to Final Concentration

The suspension concentrate in the holding vessel was diluted with water for injection, which is sterile filtered through a pre-filter and 2 serially connected sterile filters into this vessel via the milling chamber and the 70 μm stainless steel filter. After final dilution, the vessel headspace is blanketed with nitrogen and the suspension was mixed until homogeneous.

Holding and Filling of the Final Suspension

While mixing, the suspension was aseptically transferred from the holding vessel to the time/pressure (t/p) dosing vessel, from which the suspension was filled into vials which were flushed with nitrogen, stoppered and capped with an aluminium seal with a flip-off button.

(b) Solution of Hyaluronidase (rHuPH20)

A solution of rHuPH20 was prepared by diluting rHuPH20 concentrate (1×10⁶) to 10,000 U/mL by addition of 10 mM histidine, 300 mM sorbitol, 1 mg/mL methionine, pH 5.6, 0.04% polysorbate 20 buffer.

The solution was sterile, filtered and provided in 1 mL aliquot of 10,000 U/mL filled into 2R sterile glass vials.

Procedure

Six minipigs with body weights ranging from 20 to 25 kg at the start of the study were used. The minipigs were fasted overnight before dosing. Three minipigs were dosed subcutaneously in the loin with 0.19 mL of the hyaluronidase solution (10,000 U/mL) followed by 900 mg/3 mL of the drug nanosuspension at the same injection site (treatment group A). Three minipigs were dosed subcutaneously in the loin with the 900 mg/3 mL of the control drug suspension (treatment group B—control). The injection volume was 3 mL drug suspension in both treatment groups.

Method—Sequential Administration
1. Flip-off rHuPH20 solution vial flip cap and wipe with isopropyl alcohol wipe. Allow to dry. Attach an 18G transfer needle to a 1 mL syringe.
2. Draw 0.35 mL into the syringe.
3. Prime the syringe and set liquid level to 0.25 mL in the syringe.
4. Remove transfer needle and attach syringe cap to 1 mL syringe.
5. Mix rilpivirine by horizontally shaking the container 30 times over approximately 25 cm within approximately 10 s (a back and forth arm movement=2 times). Ensure well mixed/fully re-supended.
6. Flip-off rilpivirine vial flip cap and wipe with isopropyl alcohol wipe. Allow to dry.
7. Attach 18G transfer needle to 5 mL syringe.
8. Invert vial and draw >3.2 mL into 5 mL syringe mL (or as much as can be removed from vial). Injecting 1-2 mL of air will facilitate draw.
9. Detach needle and attach a syringe cap to 5 mL syringe
10. Invert syringe. Wait 5 minutes for bubbles to settle.
11. Attach a winged infusion set to the rHuPH20 1 mL syringe. Invert syringe and prime off air so that liquid forms at the tip of the needle. (0.19 mL of rHuPH20 should be in the line)
12. Insert the winged infusion set into the subcutaneous tissue of the target injection site by pinching the skin and inserting the needle at a 30-45 degree angle.
13. Release the pinch.
14. Unscrew rHuPH20 syringe from infusion set maintaining the needle in the skin. Keep luer end (open end) pointed upwards so the liquid does not drain out of the infusion line while preparing the rilpivirine syringe. It is advisable to have this syringe prepared while the technician is inserting the rHuPH20 infusion line.
15. Remove the syringe cap from the 5 mL syringe with rilpivirine. Remove air and set the dose at 3.2 mL.
16. Attach rilpivirine filled syringe to the open end of the infusion set.
17. Inject over 1 minute at a constant rate until the syringe plunger bottoms out (this will leave approximately 0.19 mL of rilpivirine in infusion line)
18. Remove the winged infusion set and dispose.
19. Record any site leakage.

Photography of Injection Site
Injection site protrusions were assessed visually.

Blood Sampling
Blood samples of 2 mL were taken from the jugular vein from all minipigs at time intervals over the following 2160 hours. Blood samples were placed on EDTA. Within 1 hour of blood sampling, samples were centrifuged at 5° C. at about 1900×g for ±10 minutes to allow plasma separation. Plasma was immediately transferred into a second tube and stored in the freezer within 1 hour after the start of centrifugation. Plasma samples were analysed individually by means of a validated LC-MS/MS method.

Pharmacokinetic Data Analysis
The pharmacokinetic profile of the blood plasma samples was evaluated using non-compartmental pharmacokinetic analysis (using individual Cₚ vs time profiles). Mean plasma concentrations and PK parameters (Cₘₐₓ, Tₘₐₓ, t_{1/2} and AUC values) were measured and the results are provided in Table 1.

### Results and Discussion

TABLE 1
Pharmacokinetic parameters

| | rHuPH20 + rilpivirine | | Rilpivirine | |
|---|---|---|---|---|
| | (treatment group A) | | (treatment group B) | |
| Parameter | according to the invention | | control | |
| N | 3 | | 3 | |
| Cₘₐₓ (ng/mL) | 52.2 ± 24.1 | | 28.5 ± 9.56 | |
| Tₘₐₓ^{a} (h) | 312 | (24-312) | 744 | (312-1248) |
| Tₗₐₛₜ^{a} (h) | 2160 | (2160-2160) | 2160 | (2160-2160) |

| | | |
|---|---|---|
| AUCₗₐₛₜ (ng*h/mL) | 24900 ± 7840 | 22330 ± 2930 |
| AUC_{∞} (ng*h/mL) | 31200^{b} | 27000 ± 3200 |
| λ_{z} (1/h) | 0.0008 ± 0.0002 | 0.0009 ± 0.00009 |

^{a}Median (Min-Max)
^{b}N = 2, SUBJECT 0005 not included in calculation of summary statistics

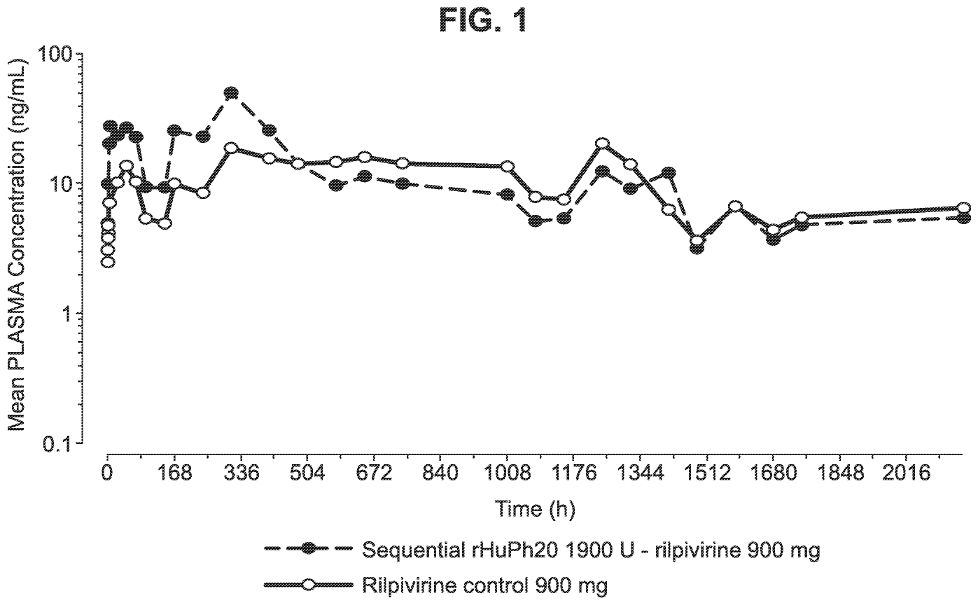

Table 1 and FIG. 1 demonstrate that administration of a hyaluronidase and a nanosuspension of a drug according to the invention results in blood plasma levels of drug over a period of at least 3 months. Surprisingly a prolonged, extended, sustained release profile of the drug is maintained when administered with the hyaluronidase.

### Example 2—Effects of Sequential and Admixed Administration of Rilpivirine with a Hyaluronidase Over 6 Months after Single Administration

This example compares the plasma kinetics, over a period of 6 months, for the following three conditions (i) administration of a suspension of rilpivirine (control), (ii) sequential administration of first a hyaluronidase solution then a rilpivirine suspension and (iii) admixed administration of a hyaluronidase solution and a rilpivirine suspension.

Preparation of Rilpivirine and Hyaluronidase Compositions

(a) Suspension of Rilpivirine

The suspension of rilpivirine was prepared as described in Example 1.

(b) Solution of Hyaluronidase (rHuPH20)

The solution of hyaluronidase was prepared as described in Example 1.

Procedure

Nine minipigs with body weights ranging from 17 to 21 kg at the start of the study were used. The minipigs were fasted overnight before dosing. The minipigs were anaesthetized with propofol before dosing. Three minipigs were dosed subcutaneously in the loin with 0.44 mL of the hyaluronidase solution (10,000 U/mL) followed by 1818 mg/6.06 mL of the rilpivirine nanosuspension at the same injection site (treatment group A—sequential). Three minipigs were dosed subcutaneously in the loin with the 1816 mg/6.5 mL admixed hyaluronidase solution (10,000 U/mL)+rilpivirine suspension (treatment group B—admixed). Three minipigs were dosed subcutaneously in the loin with the 1830 mg/6.1 mL of the control rilpivirine suspension (treatment group C—control). Vetbond 3M surgical sealant was used to seal the injection site to limit any leakage if necessary.

Method—Rilpivirine Control

The control rilpivirine suspension was prepared and administered by the following method.
1. Mix rilpivirine by horizontally shaking the container 30 times over approximately 25 cm within approximately 10 s (a back and forth arm movement=2 times). Ensure well mixed/fully re-suspended.
2. Flip-off rilpivirine vial flip cap and wipe with isopropyl alcohol wipe. Allow to dry.
3. Repeat steps 1-2 with a 2nd vial of rilpivirine. If there is a low draw, or unexpected amount of air/settling after draw, a 3rd vial may need to be prepared to ensure the proper dose level can be filled into the syringe.
4. Attach 18G transfer needle to 10 mL syringe.
5. Invert vial and draw >3.2 mL into 10 mL syringe (or as much as can be removed from vial). Injecting 1-2 mL of air will facilitate draw.
   Repeat step 5 with the 2nd vial so that at approximately 6.5 mL of drug product is in the 10 mL syringe. Important: see note in (step 3) about preparing a 3rd vial in case of low volume draw.
6. Detach needle and attach a syringe cap to 10 mL syringe
7. Wait 5 minutes for bubbles to settle with syringe inverted.
8. Remove syringe cap, invert syringe prime off air.
9. Attach a winged infusion set.
10. Set dose at 6.1 mL after filling infusion set line until liquid forms at tip of needle (0.44 mL undeliverable/dead volume will be in infusion set).
11. Insert the winged infusion set into the subcutaneous tissue of the target injection site by pinching the skin and inserting the needle at a 30-45 degree angle.
12. Release the pinch.
13. Inject over 2 minutes at a constant rate until the syringe plunger bottoms out
14. Remove the winged infusion set and dispose.
15. Record any site leakage.

Method—(i) Sequential Administration

The sequential administration of hyaluronidase solution and then rilpivirine suspension was performed according to the following method.
1. Flip-off rHuPH20 solution vial flip cap and wipe with isopropyl alcohol wipe. Swirl vial. Allow vial stopper to dry. Attach a 18G transfer needle to a 1 mL syringe.
2. Draw 0.70 mL into the syringe.
3. Prime the syringe and set liquid level to 0.60 mL in the syringe.
4. Remove transfer needle and attach syringe cap to 1 mL syringe.
5. Mix rilpivirine by horizontally shaking the container 30 times over approximately 25 cm within approximately 10 s (a back and forth arm movement=2 times). Ensure well mixed/fully resuspended.
6. Flip-off rilpivirine vial flip cap and wipe with isopropyl alcohol wipe. Allow to dry.
7. Repeat steps 5-6 with a 2nd vial of rilpivirine. If there is a low draw, or unexpected amount of air/settling after draw, a 3rd vial may need to be prepared to ensure the proper dose level can be filled into the syringe.
8. Attach 18G transfer needle to 10 mL syringe.
9. Invert vial and draw >3.2 mL into 10 mL syringe (or as much as can be removed from vial). Injecting 1-2 mL of air will facilitate draw.
10. Repeat step 9 with the 2nd vial so that at approximately 6.5 mL of drug product is in the 10 mL syringe. Important: see note in (step 7) about preparing a 3rd vial in case of low volume draw.
11. Detach needle and attach a syringe cap to 10 mL syringe
12. Invert syringe. Wait 5 minutes for bubbles to settle.
13. Attach a winged infusion set to the rHuPH20 1 mL syringe. Invert syringe and prime off air so that liquid forms at the tip of the needle. (0.44 mL of rHuPH20 should be in the line)
14. Insert the winged infusion set into the subcutaneous tissue of the target injection site by pinching the skin and inserting the needle at a 30-45 degree angle.
15. Release the pinch.
16. Unscrew rHuPH20 syringe from infusion set maintaining the needle in the skin. Keep open end pointed upwards so the liquid does not drain out of the infusion line while preparing the rilpivirine syringe. It is advisable to have this syringe prepared while the technician is inserting the rHuPH20 infusion line.
17. Remove the syringe cap from the 10 mL syringe with rilpivirine. Remove air and set the dose at approximately 6.5 mL.
18. Attach rilpivirine filled syringe to the open end of the infusion set.
19. Inject over 1 minute at a constant rate until the syringe plunger bottoms out (this will leave approximately 0.44 mL of rilpivirine in infusion line)
20. Remove the winged infusion set and dispose.
21. Record any site leakage.

Method—(ii) Admixed Administration

The admixed administration of hyaluronidase solution and rilpivirine suspension was performed according to the following method.
1. Flip-off rHuPH20 solution vial flip cap and wipe with isopropyl alcohol wipe. Allow to dry. Attach a 18G transfer needle to a 1 mL syringe.
2. Draw 0.40 mL into the syringe.
3. Prime the syringe and set liquid level to 0.35 mL in the syringe.
4. Remove transfer needle and attach syringe cap to 1 mL syringe.
5. Mix rilpivirine by horizontally shaking the container 30 times over approximately 25 cm within approximately 10s (a back and forth arm movement=2 times). Ensure well mixed/fully resuspended.
6. Flip-off rilpivirine vial flip cap and wipe with isopropyl alcohol wipe. Allow to dry.
7. Remove syringe cap from 1 mL PH20 filled syringe and attach 25G needle.
8. Prime the syringe so that liquid forms at needle tip and the syringe is set at approximately 0.25 mL
9. Insert 25 G needle/rHuPH20 solution syringe into vial so that the needle is in the liquid.
10. Transfer 0.25 mL of rHuPH20 solution (2500 U) into rilpivirine vial.
11. Shake vial gently.
12. Repeat steps 1-10 to prepare a second vial of rilpivirine with rHuPH20. If there is a low draw, or unexpected amount of air/settling after draw, a 3rd vial may need to be prepared to ensure the proper dose level can be filled into the syringe.
13. Attach 18G transfer needle to 10 mL syringe.
14. Invert vial and draw >3.4 mL (or as much as can be removed from vial) into 10 mL syringe. Injecting 1-2 mL of air will facilitate draw.
15. Repeat step 14 with the 2nd prepared vial so that at approximately 7.0 mL of drug product is in the 10 mL syringe. Important: see note in (step 12) about preparing a 3rd vial in case of low volume draw.
16. Detach needle and attach a syringe cap to 10 mL syringe
17. Invert syringe and wait 5 minutes for bubbles to settle.
18. Remove syringe cap and prime off air so that a drop of liquid is at the needle, set the dose to 6.5 mL after priming.
19. Attach a winged infusion set to the 10 mL syringe. Invert syringe and prime off air so that liquid forms at the tip of the needle.
20. Insert the winged infusion set into the subcutaneous tissue of the target injection site by pinching the skin and inserting the needle at a 30-45 degree angle.
21. Inject over 1 minute at a constant rate until the syringe plunger bottoms out (this will leave approximately 0.44 mL of rilpivirine in infusion line)
22. Remove the winged infusion set and dispose.
23. Record any site leakage.

Photography of Injection Site
Injection site protrusions were assessed visually.

Blood Sampling
Blood samples of 2 mL were taken from the jugular vein from all minipigs at time intervals over the following 6 months. Blood samples were placed on EDTA. Within 1 hour of blood sampling, samples were centrifuged at 5° C. at about 1900×g for ±10 minutes to allow plasma separation. Plasma was immediately transferred into a second tube and stored in the freezer within 1 hour after the start of centrifugation. Plasma samples were analysed individually by means of a validated LC-MS/MS method.

Pharmacokinetic Data Analysis
The PK profiles of the blood plasma samples was evaluated using non-compartmental pharmacokinetic analysis (using individual Cₚ vs time profiles). Mean plasma concentrations and PK parameters (Cₘₐₓ and AUC values) were measured and the results are provided in Table 2.

### Results and Discussion

PK parameters after single subcutaneous administration of rilpivirine nanosuspension at 6 mL with (sequential and admixed administration) and without rHuPH20 solution are shown in Table 2.

TABLE 2
Pharmacokinetic parameters

| | rHuPH20 + | rHuPH20 + | |
|---|---|---|---|
| | rilpivirine sequential | rilpivirine admixed | Rilpivirine |
| | (treatment | (treatment | (treatment |
| | group A) according | group B) according | group C) |
| | to the invention | to the invention | control |
| N | 3 | 3 | 3 |
| Mean Cₘₐₓ | 146 | 94 | 49^{a} |
| (ng/mL) | | | |
| Mean | 38400 | 21000 | 22000 |
| AUC_{0-1 months} | | | |
| (ng*h/mL) | | | |
| Mean | 136000 | 107000 | 78300 |
| AUC_{0-6 months} | | | |
| (ng*h/mL) | | | |

^{a}Excluding an outlier minipig (with a Cₘₐₓ of 563 ng/mL at 7 hours post-administration).

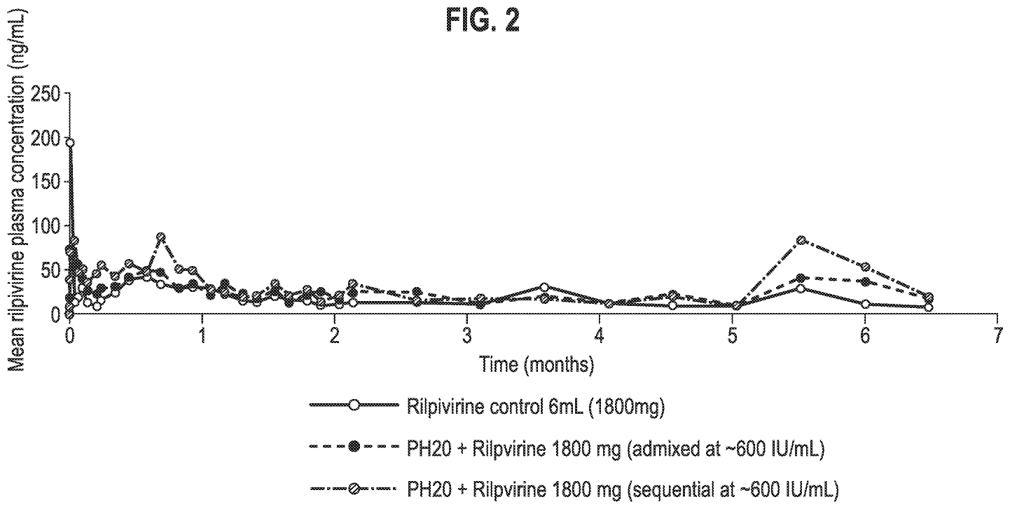

Table 2 and FIG. 2 demonstrate that both sequential and admixed administration of a hyaluronidase and a nanosuspension of rilpivirine according to the invention and administration of a nanosuspension of rilpivirine alone resulted in slow release from the injection site with measurable blood plasma levels of rilpivirine over a period of at least 6 months. Surprisingly a prolonged, extended, sustained release profile of rilpivirine is maintained when administered with the hyaluronidase both sequentially and after admixed administration.

Also described herein are the following numbered clauses.

1. A drug and a hyaluronidase for use in therapy,
wherein the drug is in the form of micro- or nanoparticles in suspension,
wherein the drug and hyaluronidase are administered by subcutaneous or intramuscular injection, and
wherein the drug and hyaluronidase are administered intermittently at a time interval of about three months to about two years.

2. The drug and a hyaluronidase for use according to clause 1, wherein the hyaluronidase is recombinant human hyaluronidase (e.g. rHuPH20), for example comprising the amino acid sequence of SEQ ID NO: 1.

3. The drug and a hyaluronidase for use according to any one of the preceding clauses, wherein the time interval is about three months to about one year, in particular wherein the time interval is about six months.

4. The drug and a hyaluronidase for use according to clause 3, wherein the time interval is about three months to about six months.

5. The drug and a hyaluronidase for use according to clause 4, wherein the time interval is about six months to about one year.

6. The drug and a hyaluronidase for use according to any one of the preceding clauses, wherein the drug and hyaluronidase are administered simultaneously or sequentially.

7. The drug and a hyaluronidase for use according to any one of the preceding clauses, wherein the micro- or nanoparticles have a surface modifier adsorbed to their surface.

8. The drug and a hyaluronidase for use according to clause 7, wherein the surface modifier is a poloxamer or a polysorbate.

9. The drug and a hyaluronidase for use according to clause 8, wherein the surface modifier is a poloxamer which is poloxamer 338 or wherein the surface modifier is a polysorbate.

10. The drug and a hyaluronidase for use according to any one of the preceding clauses, wherein the average effective particle size of the micro- or nanoparticles is less than about 20 μm.

11. The drug and a hyaluronidase for use according to clause 10, wherein the average effective particle size of the micro- or nanoparticles is less than about 10 μm.

12. The drug and a hyaluronidase for use according to clause 11, wherein the average effective particle size of the micro- or nanoparticles is from about 25 nm to about 10 μm.

13. The drug and a hyaluronidase for use according to clause 12, wherein the average effective particle size of the micro- or nanoparticles is from about 200 nm to about 10 μm.

14. The drug and a hyaluronidase for use according to clause 13, wherein the average effective particle size of the micro- or nanoparticles is from about 200 nm to about 5 μm.

15. The drug and a hyaluronidase for use according to any one of the preceding clauses, wherein the drug and the hyaluronidase are administered sequentially.

16. The drug and a hyaluronidase for use according to any one of the preceding clauses, wherein the drug and hyaluronidase are administered in separate pharmaceutical compositions.

17. The drug and a hyaluronidase for use according to clause 16, wherein the pharmaceutical composition comprising the hyaluronidase is a solution, and the concentration of the hyaluronidase in the solution is from about 50 to about 10,000 U/mL, in particular is about 2,000 U/mL.

18. The drug and a hyaluronidase for use according to any one of clauses 1-14, wherein the drug and the hyaluronidase are administered as a combined pharmaceutical composition.

19. The drug and a hyaluronidase for use according to any one of the preceding clauses, wherein the drug is selected from: drugs for treatment of chronic and long-term diseases and disorders, for example for treatment of chronic viral infection (such as chronic infection with Varicella-zoster virus, measles virus, HIV, hepatitis B virus, hepatitis C virus, hepatitis D virus or human cytomegalovirus), cancer, psychiatric diseases and disorders, mood disorders (such as bipolar, cyclothymic or depression), diabetes, hypertension, abnormal cholesterol and triglyceride levels, inflammatory disorders (such as allergy, asthma, autoimmune diseases, coeliac disease, hepatitis, inflammatory bowel disease, Crohn disease, gout, myositis, scleroderma, rheumatoid arthritis, lupus vasculitis, ankylosing spondylitis or chronic obstructive pulmonary disease), cystic fibrosis, multiple sclerosis, autoimmune disorders, neurodegenerative disorders (such as Parkinson Disease or Alzheimer disease), chronic pain, inherited metabolic disorders or epilepsy.

20. The drug and a hyaluronidase for use according to any one of the preceding clauses, wherein the drug is selected from rilpivirine (TMC278), apalutamide, enzalutamide, and darolutamide, or pharmaceutically acceptable salts thereof.

21. The drug and a hyaluronidase for use according to any one of the preceding clauses, wherein the drug is rilpivirine or a pharmaceutically acceptable salt thereof.

22. The drug and a hyaluronidase for use according to clause 21, wherein the drug is rilpivirine.

23. The drug and a hyaluronidase for use according to any one of the preceding clauses, wherein the drug is not an antibody.

24. The drug and a hyaluronidase for use according to any one of the preceding clauses, wherein the drug is not a biologic.

25. The drug and a hyaluronidase for use according to any one of the preceding clauses, wherein the drug has a molecular weight of less than 1000 Da.

26. The drug and a hyaluronidase for use according to any one of the preceding clauses, wherein the drug and hyaluronidase are administered by subcutaneous injection.

27. The drug and a hyaluronidase for use according to any one of the preceding clauses, wherein the suspension comprises a pharmaceutically acceptable aqueous carrier in which the drug is suspended.

28. The drug and a hyaluronidase for use according to any one of the preceding clauses, wherein the disease or disorder is HIV infection.

29. The drug and a hyaluronidase for use according to clause 28, wherein the disease or disorder is HIV type 1 (HIV-1) infection.

30. The drug and a hyaluronidase for use according to any one of clauses 1-27, wherein the disease or disorder is cancer.

31. The drug and a hyaluronidase for use according to any one of the preceding clauses, wherein the subject is a human.

32. A combination for use in therapy, wherein the combination comprises a drug and a hyaluronidase,
wherein the drug is in the form of micro- or nanoparticles in suspension, and
wherein the combination is administered intermittently by subcutaneous or intramuscular injection at a time interval of about three months to about two years.

33. The combination for use according to clause 32, wherein the hyaluronidase is recombinant human hyaluronidase (e.g. rHuPH20), for example comprising the amino acid sequence of SEQ ID NO: 1.

34. The combination for use according to any one of clauses 32-33, wherein the time interval is about three months to about one year.

35. The combination for use according to clause 34, wherein the time interval is about three months to about six months.

36. The combination for use according to clause 35, wherein the time interval is about six months to about one year, in particular wherein the time interval is about six months.

37. The combination for use according to any one of clauses 32-36, wherein the drug and hyaluronidase are administered simultaneously or sequentially.

38. The combination for use according to any one of clauses 32-37, wherein the micro- or nanoparticles have a surface modifier adsorbed to their surface.

39. The combination for use according to clause 38, wherein the surface modifier is a poloxamer or a polysorbate.

40. The combination for use according to clause 39, wherein the surface modifier is a poloxamer which is poloxamer 338 or wherein the surface modifier is a polysorbate.

41. The combination for use according to any one of clauses 32-40, wherein the average effective particle size of the micro- or nanoparticles is less than about 20 μm.

42. The combination for use according to clause 41, wherein the average effective particle size of the micro- or nanoparticles is less than about 10 μm.

43. The combination for use according to clause 42, wherein the average effective particle size of the micro- or nanoparticles is from about 25 nm to about 10 μm.

44. The combination for use according to clause 43, wherein the average effective particle size of the micro- or nanoparticles is from about 200 nm to about 10 μm.

45. The combination for use according to clause 44, wherein the average effective particle size of the micro- or nanoparticles is from about 200 nm to about 5 μm.

46. The combination for use according to any one of clauses 32-45, wherein the drug and the hyaluronidase are administered sequentially.

47. The combination for use according to any one of clauses 32-46, wherein the drug and hyaluronidase are administered in separate pharmaceutical compositions.

48. The combination for use according to clause 47, wherein the pharmaceutical composition comprising the hyaluronidase is a solution, and the concentration of the hyaluronidase in the solution is from about 50 to about 10,000 U/mL, in particular is about 2,000 U/mL.

49. The combination for use according to any one of clauses 32-45, wherein the drug and the hyaluronidase are administered as a combined pharmaceutical composition.

50. The combination for use according to any one of clauses 32-49, wherein the drug is selected from: drugs for treatment of chronic and long-term diseases and disorders, for example for treatment of chronic viral infection (such as chronic infection with Varicella-zoster virus, measles virus, HIV, hepatitis B virus, hepatitis C virus, hepatitis D virus or human cytomegalovirus), cancer, psychiatric diseases and disorders, mood disorders (such as bipolar, cyclothymic or depression), diabetes, hypertension, abnormal cholesterol and triglyceride levels, inflammatory disorders (such as allergy, asthma, autoimmune diseases, coeliac disease, hepatitis, inflammatory bowel disease, Crohn disease, gout, myositis, scleroderma, rheumatoid arthritis, lupus vasculitis, ankylosing spondylitis or chronic obstructive pulmonary disease), cystic fibrosis, multiple sclerosis, autoimmune disorders, neurodegenerative disorders (such as Parkinson Disease or Alzheimer disease), chronic pain, inherited metabolic disorders or epilepsy.

51. The combination for use according to any one of clauses 32-50, wherein the drug is selected from selected from rilpivirine (TMC278), apalutamide, enzalutamide, and darolutamide, or pharmaceutically acceptable salts thereof.

52. The combination for use according to any one of clauses 32-51, wherein the drug is rilpivirine or a pharmaceutically acceptable salt thereof.

53. The combination for use according to clause 52, wherein the drug is rilpivirine.

54. The combination for use according to any one of clauses 32-53, wherein the drug is not an antibody.

55. The combination for use according to any one of clauses 32-54, wherein the drug is not a biologic.

56. The combination for use according to any one of clauses 32-55, wherein the drug has a molecular weight of less than 1000 Da.

57. The combination for use according to any one of clauses 32-56, wherein the drug and hyaluronidase are administered by subcutaneous injection.

58. The combination for use according to any one of clauses 32-57, wherein the suspension comprises a pharmaceutically acceptable aqueous carrier in which the drug is suspended.

59. The combination for use according to any one of clauses 32-58, wherein the disease or disorder is HIV infection.

60. The combination for use according to clause 59, wherein the disease or disorder is HIV type 1 (HIV-1) infection.

61. The combination for use according to any one of clauses 32-58, wherein the disease or disorder is cancer.

62. The combination for use according to any one of clauses 32-61, wherein the subject is a human.

63. Products containing a drug and a hyaluronidase as a combined preparation for simultaneous or sequential use in therapy by subcutaneous or intramuscular injection,
wherein the drug is in the form of micro- or nanoparticles in suspension, and
wherein the drug and the hyaluronidase are administered intermittently at a time interval of about three months to about two years.

64. The products for simultaneous or sequential use according to clause 63, wherein the hyaluronidase is recombinant human hyaluronidase (e.g. rHuPH20), for example comprising the amino acid sequence of SEQ ID NO: 1.

65. The products for simultaneous or sequential use according to any one of clauses 63-64, wherein the time interval is about three months to about one year.

66. The products for simultaneous or sequential use according to clause 65, wherein the time interval is about three months to about six months.

67. The products for simultaneous or sequential use according to clause 66, wherein the time interval is about six months to about one year, in particular wherein the time interval is about six months.

68. The products for simultaneous or sequential use according to any one of clauses 63-67, wherein the drug and hyaluronidase are administered sequentially.

69. The products for simultaneous or sequential use according to any one of clauses 63-68, wherein the micro- or nanoparticles have a surface modifier adsorbed to their surface.

70. The products for simultaneous or sequential use according to clause 69, wherein the surface modifier is a poloxamer or a polysorbate.

71. The products for simultaneous or sequential use according to clause 70, wherein the surface modifier is a poloxamer which is poloxamer 338 or wherein the surface modifier is a polysorbate.

72. The products for simultaneous or sequential use according to any one of clauses 63-71, wherein the average effective particle size of the micro- or nanoparticles is less than about 20 μm.

73. The products for simultaneous or sequential use according to clause 72, wherein the average effective particle size of the micro- or nanoparticles is less than about 10 μm.

74. The products for simultaneous or sequential use according to clause 73, wherein the average effective particle size of the micro- or nanoparticles is from about 25 nm to about 10 μm.

75. The products for simultaneous or sequential use according to clause 74, wherein the average effective particle size of the micro- or nanoparticles is from about 200 nm to about 10 μm.

76. The products for simultaneous or sequential use according to clause 75, wherein the average effective particle size of the micro- or nanoparticles is from about 200 nm to about 5 μm.

77. The products for simultaneous or sequential use according to any one of clauses 63-76, wherein the drug and the hyaluronidase are administered sequentially by subcutaneous injection.

78. The products for simultaneous or sequential use according to any one of clauses 63-77, wherein the drug and hyaluronidase are administered in separate pharmaceutical compositions.

79. The products for simultaneous or sequential use according to clause 78, wherein the pharmaceutical composition comprising the hyaluronidase is a solution, and the concentration of the hyaluronidase in the solution is from about 50 to about 10,000 U/mL, in particular is about 2,000 U/mL.

80. The products for simultaneous or sequential use according to any one of clauses 63-76, wherein the drug and the hyaluronidase are administered as a combined pharmaceutical composition.

81. The products for simultaneous or sequential use according to any one of clauses 63-80, wherein the drug is selected from: drugs for treatment of chronic and long-term diseases and disorders, for example for treatment of chronic viral infection (such as chronic infection with Varicella-zoster virus, measles virus, HIV, hepatitis B virus, hepatitis C virus, hepatitis D virus or human cytomegalovirus), cancer, psychiatric diseases and disorders, mood disorders (such as bipolar, cyclothymic or depression), diabetes, hypertension, abnormal cholesterol and triglyceride levels, inflammatory disorders (such as allergy, asthma, autoimmune diseases, coeliac disease, hepatitis, inflammatory bowel disease, Crohn disease, gout, myositis, scleroderma, rheumatoid arthritis, lupus vasculitis, ankylosing spondylitis or chronic obstructive pulmonary disease), cystic fibrosis, multiple sclerosis, autoimmune disorders, neurodegenerative disorders (such as Parkinson Disease or Alzheimer disease), chronic pain, inherited metabolic disorders or epilepsy.

82. The products for simultaneous or sequential use according to any one of clauses 63-81, wherein the drug is selected from rilpivirine (TMC278), apalutamide, enzalutamide, and darolutamide, or pharmaceutically acceptable salts thereof.

83. The products for simultaneous or sequential use according to any one of clauses 63-82, wherein the drug is rilpivirine or a pharmaceutically acceptable salt thereof.

84. The products for simultaneous or sequential use according to clause 83, wherein the drug is rilpivirine.

85. The products for simultaneous or sequential use according to any one of clauses 63-84, wherein the drug is not an antibody.

86. The products for simultaneous or sequential use according to any one of clauses 63-85, wherein the drug is not a biologic.

87. The products for simultaneous or sequential use according to any one of clauses 63-86, wherein the drug has a molecular weight of less than 1000 Da.

88. The products for simultaneous or sequential use according to any one of clauses 63-87, wherein the drug and hyaluronidase are administered by subcutaneous injection.

89. The products for simultaneous or sequential use according to any one of clauses 63-88, wherein the suspension comprises a pharmaceutically acceptable aqueous carrier in which the drug is suspended.

90. The products for simultaneous or sequential use according to any one of clauses 63-89, wherein the disease or disorder is HIV infection.

91. The products for simultaneous or sequential use according to clause 90, wherein the disease or disorder is HIV type 1 (HIV-1) infection.

92. The products for simultaneous or sequential use according to any one of clauses 63-89, wherein the disease or disorder is cancer.

93. The products for simultaneous or sequential use according to any one of clauses 63-92, wherein the subject is a human.

94. A kit of parts comprising a drug and a hyaluronidase for simultaneous or sequential use in therapy by subcutaneous or intramuscular injection,
wherein the drug is in the form of micro- or nanoparticles in suspension, and
wherein the drug and the hyaluronidase are administered intermittently at a time interval of about three months to about two years.

95. The kit of parts for simultaneous or sequential use according to clause 94, wherein the hyaluronidase is recombinant human hyaluronidase (e.g. rHuPH20), for example comprising the amino acid sequence of SEQ ID NO: 1.

96. The kit of parts for simultaneous or sequential use according to any one of clauses 94-95, wherein the time interval is about three months to about one year.

97. The kit of parts for simultaneous or sequential use according to clause 96, wherein the time interval is about three months to about six months.

98. The kit of parts for simultaneous or sequential use according to clause 97, wherein the time interval is about six months to about one year, in particular wherein the time interval is about six months.

99. The kit of parts for simultaneous or sequential use according to any one of clauses 94-98, wherein the drug and hyaluronidase are administered sequentially.

100. The kit of parts for simultaneous or sequential use according to any one of clauses 94-99, wherein the micro- or nanoparticles have a surface modifier adsorbed to their surface.

101. The kit of parts for simultaneous or sequential use according to clause 100, wherein the surface modifier is a poloxamer or a polysorbate.

102. The kit of parts for simultaneous or sequential use according to clause 101, wherein the surface modifier is a poloxamer which is poloxamer 338 or wherein the surface modifier is a polysorbate.

103. The kit of parts for simultaneous or sequential use according to any one of clauses 94-102, wherein the average effective particle size of the micro- or nanoparticles is less than about 20 μm.

104. The kit of parts for simultaneous or sequential use according to clause 103, wherein the average effective particle size of the micro- or nanoparticles is less than about 10 μm.

105. The kit of parts for simultaneous or sequential use according to clause 104, wherein the average effective particle size of the micro- or nanoparticles is from about 25 nm to about 10 μm.

106. The kit of parts for simultaneous or sequential use according to clause 105, wherein the average effective particle size of the micro- or nanoparticles is from about 200 nm to about 10 μm.

107. The kit of parts for simultaneous or sequential use according to clause 106, wherein the average effective particle size of the micro- or nanoparticles is from about 200 nm to about 5 μm.

108. The kit of parts for simultaneous or sequential use according to any one of clauses 94-107, wherein the drug and the hyaluronidase are administered sequentially by subcutaneous injection.

109. The kit of parts for simultaneous or sequential use according to any one of clauses 94-108, wherein the drug and hyaluronidase are administered in separate pharmaceutical compositions.

110. The kit of parts for simultaneous or sequential use according to clause 109, wherein the pharmaceutical composition comprising the hyaluronidase is a solution, and the concentration of the hyaluronidase in the solution is from about 50 to about 10,000 U/mL, in particular is about 2,000 U/mL.

111. The kit of parts for simultaneous or sequential use according to any one of clauses 94-107, wherein the drug and the hyaluronidase are administered as a combined pharmaceutical composition.

112. The kit of parts for simultaneous or sequential use according to any one of clauses 94-111, wherein the drug is selected from: drugs for treatment of chronic and long-term diseases and disorders, for example for treatment of chronic viral infection (such as chronic infection with Varicella-zoster virus, measles virus, HIV, hepatitis B virus, hepatitis C virus, hepatitis D virus or human cytomegalovirus), cancer, psychiatric diseases and disorders, mood disorders (such as bipolar, cyclothymic or depression), diabetes, hypertension, abnormal cholesterol and triglyceride levels, inflammatory disorders (such as allergy, asthma, autoimmune diseases, coeliac disease, hepatitis, inflammatory bowel disease, Crohn disease, gout, myositis, scleroderma, rheumatoid arthritis, lupus vasculitis, ankylosing spondylitis or chronic obstructive pulmonary disease), cystic fibrosis, multiple sclerosis, autoimmune disorders, neurodegenerative disorders (such as Parkinson Disease or Alzheimer disease), chronic pain, inherited metabolic disorders or epilepsy.

113. The kit of parts for simultaneous or sequential use according to any one of clauses 94-112, wherein the drug is selected from rilpivirine (TMC278), apalutamide, enzalutamide, and darolutamide, or pharmaceutically acceptable salts thereof.

114. The kit of parts for simultaneous or sequential use according to any one of clauses 94-113, wherein the drug is rilpivirine or a pharmaceutically acceptable salt thereof.

115. The kit of parts for simultaneous or sequential use according to clause 114, wherein the drug is rilpivirine.

116. The kit of parts for simultaneous or sequential use according to any one of clauses 94-115, wherein the drug is not an antibody.

117. The kit of parts for simultaneous or sequential use according to any one of clauses 94-116, wherein the drug is not a biologic.

118. The kit of parts for simultaneous or sequential use according to any one of clauses 94-117, wherein the drug has a molecular weight of less than 1000 Da.

119. The kit of parts for simultaneous or sequential use according to any one of clauses 94-118, wherein the drug and hyaluronidase are administered by subcutaneous injection.

120. The kit of parts for simultaneous or sequential use according to any one of clauses 94-119, wherein the suspension comprises a pharmaceutically acceptable aqueous carrier in which the drug is suspended.

121. The kit of parts for simultaneous or sequential use according to any one of clauses 94-120, wherein the disease or disorder is HIV infection.

122. The kit of parts for simultaneous or sequential use according to clause 121, wherein the disease or disorder is HIV type 1 (HIV-1) infection.

123. The kit of parts for simultaneous or sequential use according to any one of clauses 94-120, wherein the disease or disorder is cancer.

124. The kit of parts for simultaneous or sequential use according to any one of clauses 94-123, wherein the subject is a human.

125. A drug in the form of micro- or nanoparticles in suspension for use in therapy by subcutaneous or intramuscular injection,
wherein the drug is administered in combination with a hyaluronidase that is administered by subcutaneous or intramuscular injection, and
wherein the drug and the hyaluronidase are administered intermittently at a time interval of about three months to about two years.

126. The drug for use according to clause 125, wherein the hyaluronidase is recombinant human hyaluronidase (e.g. rHuPH20), for example comprising the amino acid sequence of SEQ ID NO: 1.

127. The drug for use according to any one of clauses 125-126, wherein the time interval is about three months to about one year.

128. The drug for use according to clause 127, wherein the time interval is about three months to about six months, in particular wherein the time interval is about six months.

129. The drug for use according to clause 128, wherein the time interval is about six months to about one year.

130. The drug for use according to any one of clauses 125-129, wherein the drug and hyaluronidase are administered simultaneously or sequentially.

131. The drug for use according to any one of clauses 125-130, wherein the micro- or nanoparticles have a surface modifier adsorbed to their surface.

132. The drug for use according to clause 131, wherein the surface modifier is a poloxamer or a polysorbate.

133. The drug for use according to clause 132, wherein the surface modifier is a poloxamer which is poloxamer 338 or wherein the surface modifier is a polysorbate.

134. The drug for use according to any one of clauses 125-133, wherein the average effective particle size of the micro- or nanoparticles is less than about 20 μm.

135. The drug for use according to clause 134, wherein the average effective particle size of the micro- or nanoparticles is less than about 10 μm.

136. The drug for use according to clause 135, wherein the average effective particle size of the micro- or nanoparticles is from about 25 nm to about 10 μm.

137. The drug for use according to clause 136, wherein the average effective particle size of the micro- or nanoparticles is from about 200 nm to about 10 μm.

138. The drug for use according to clause 137, wherein the average effective particle size of the micro- or nanoparticles is from about 200 nm to about 5 μm.

139. The drug for use according to any one of clauses 125-138, wherein the drug and the hyaluronidase are administered sequentially.

140. The drug for use according to any one of clauses 125-139, wherein the drug and hyaluronidase are administered in separate pharmaceutical compositions.

141. The drug for use according to clause 140, wherein the pharmaceutical composition comprising the hyaluronidase is a solution, and the concentration of the hyaluronidase in the solution is from about 50 to about 10,000 U/mL, in particular is about 2,000 U/mL.

142. The drug for use according to any one of clauses 125-138, wherein the drug and the hyaluronidase are administered as a combined pharmaceutical composition.

143. The drug for use according to any one of clauses 125-142, wherein the drug is selected from: drugs for treatment of chronic and long-term diseases and disorders, for example for treatment of chronic viral infection (such as chronic infection with Varicella-zoster virus, measles virus, HIV, hepatitis B virus, hepatitis C virus, hepatitis D virus or human cytomegalovirus), cancer, psychiatric diseases and disorders, mood disorders (such as bipolar, cyclothymic or depression), diabetes, hypertension, abnormal cholesterol and triglyceride levels, inflammatory disorders (such as allergy, asthma, autoimmune diseases, coeliac disease, hepatitis, inflammatory bowel disease, Crohn disease, gout, myositis, scleroderma, rheumatoid arthritis, lupus vasculitis, ankylosing spondylitis or chronic obstructive pulmonary disease), cystic fibrosis, multiple sclerosis, autoimmune disorders, neurodegenerative disorders (such as Parkinson Disease or Alzheimer disease), chronic pain, inherited metabolic disorders or epilepsy.

144. The drug for use according to any one of clauses 125-143, wherein the drug is selected from selected from rilpivirine (TMC278), apalutamide, enzalutamide, and darolutamide, or pharmaceutically acceptable salts thereof.

145. The drug for use according to any one of clauses 125-144, wherein the drug is rilpivirine or a pharmaceutically acceptable salt thereof.

146. The drug for use according to clause 145, wherein the drug is rilpivirine.

147. The drug for use according to any one of clauses 125-146, wherein the drug is not an antibody.

148. The drug for use according to any one of clauses 125-147, wherein the drug is not a biologic.

149. The drug for use according to any one of clauses 125-148, wherein the drug has a molecular weight of less than 1000 Da.

150. The drug for use according to any one of clauses 125-149, wherein the drug and hyaluronidase are administered by subcutaneous injection.

151. The drug for use according to any one of clauses 125-150, wherein the suspension comprises a pharmaceutically acceptable aqueous carrier in which the drug is suspended.

152. The drug for use according to any one of clauses 125-151, wherein the disease or disorder is HIV infection.

153. The drug for use according to clause 152, wherein the disease or disorder is HIV type 1 (HIV-1) infection.

154. The drug for use according to any one of clauses 125-151, wherein the disease or disorder is cancer.

155. The drug for use according to any one of clauses 125-154, wherein the subject is a human.

156. Use of a drug for the manufacture of a medicament for use in the treatment of a disease or disorder in a subject,
wherein the drug is in the form of micro- or nanoparticles in suspension and is administered in combination with a hyaluronidase,
wherein the drug and the hyaluronidase are administered to the subject by subcutaneous or intramuscular injection, and
wherein the drug and the hyaluronidase are administered intermittently at a time interval of about three months to about two years.

157. The use according to clause 156, wherein the hyaluronidase is recombinant human hyaluronidase (e.g. rHuPH20), for example comprising the amino acid sequence of SEQ ID NO: 1.

158. The use according to any one of clauses 156-157, wherein the time interval is about three months to about one year, in particular wherein the time interval is about six months.

159. The use according to clause 158, wherein the time interval is about three months to about six months.

160. The use according to clause 159, wherein the time interval is about six months to about one year.

161. The use according to any one of clauses 156-159, wherein the drug and hyaluronidase are administered simultaneously or sequentially.

162. The use according to any one of clauses 156-161, wherein the micro- or nanoparticles have a surface modifier adsorbed to their surface.

163. The use according to clause 162, wherein the surface modifier is a poloxamer or a polysorbate.

164. The use according to clause 163, wherein the surface modifier is a poloxamer which is poloxamer 338 or wherein the surface modifier is a polysorbate.

165. The use according to any one of clauses 156-164, wherein the average effective particle size of the micro- or nanoparticles is less than about 20 μm.

166. The use according to clause 165, wherein the average effective particle size of the micro- or nanoparticles is less than about 10 μm.

167. The use according to clause 166, wherein the average effective particle size of the micro- or nanoparticles is from about 25 nm to about 10 μm.

168. The use according to clause 167, wherein the average effective particle size of the micro- or nanoparticles is from about 200 nm to about 10 μm.

169. The use according to clause 168, wherein the average effective particle size of the micro- or nanoparticles is from about 200 nm to about 5 μm.

170. The use according to any one of clauses 156-169, wherein the drug and the hyaluronidase are administered sequentially.

171. The use according to any one of clauses 156-170, wherein the drug and hyaluronidase are administered in separate pharmaceutical compositions.

172. The use according to clause 171, wherein the pharmaceutical composition comprising the hyaluronidase is a solution, and the concentration of the hyaluronidase in the solution is from about 50 to about 10,000 U/mL, in particular is about 2,000 U/mL.

173. The use according to any one of clauses 156-169, wherein the drug and the hyaluronidase are administered as a combined pharmaceutical composition.

174. The use according to any one of clauses 156-173, wherein the drug is selected from: drugs for treatment of chronic and long-term diseases and disorders, for example for treatment of chronic viral infection (such as chronic infection with Varicella-zoster virus, measles virus, HIV, hepatitis B virus, hepatitis C virus, hepatitis D virus or human cytomegalovirus), cancer, psychiatric diseases and disorders, mood disorders (such as bipolar, cyclothymic or depression), diabetes, hypertension, abnormal cholesterol and triglyceride levels, inflammatory disorders (such as allergy, asthma, autoimmune diseases, coeliac disease, hepatitis, inflammatory bowel disease, Crohn disease, gout, myositis, scleroderma, rheumatoid arthritis, lupus vasculitis, ankylosing spondylitis or chronic obstructive pulmonary disease), cystic fibrosis, multiple sclerosis, autoimmune disorders, neurodegenerative disorders (such as Parkinson Disease or Alzheimer disease), chronic pain, inherited metabolic disorders or epilepsy.

175. The use according to any one of clauses 156-174, wherein the drug selected from rilpivirine (TMC278), apalutamide, enzalutamide, and darolutamide, or pharmaceutically acceptable salts thereof.

176. The use according to any one of clauses 156-175, wherein the drug is rilpivirine or a pharmaceutically acceptable salt thereof.

177. The use according to clause 176, wherein the drug is rilpivirine.

178. The use according to any one of clauses 156-177, wherein the drug is not an antibody.

179. The use according to any one of clauses 156-178, wherein the drug is not a biologic.

180. The use according to any one of clauses 156-179, wherein the drug has a molecular weight of less than 1000 Da.

181. The use according to any one of clauses 156-180, wherein the drug and hyaluronidase are administered by subcutaneous injection.

182. The use according to any one of clauses 156-181, wherein the suspension comprises a pharmaceutically acceptable aqueous carrier in which the drug is suspended.

183. The use according to any one of clauses 156-182, wherein the disease or disorder is HIV infection.

184. The use according to clause 183, wherein the disease or disorder is HIV type 1 (HIV-1) infection.

185. The use according to any one of clauses 156-182, wherein the disease or disorder is cancer.

186. The use according to any one of clauses 156-185, wherein the subject is a human.

1. A method for the treatment or prevention of a disease or disorder in a subject in need thereof, the method comprising administering to the subject a drug effective in the treatment or prevention of the disease or disorder in the form of micro- or nanoparticles in suspension by intramuscular injection or subcutaneous injection,
wherein the drug is administered in combination with a hyaluronidase that is administered by intramuscular injection or subcutaneous injection, and
wherein the drug and the hyaluronidase are administered intermittently at a time interval of about three months to about two years.

2. The method according to claim 1, wherein the hyaluronidase is recombinant human hyaluronidase (e.g. rHuPH20), for example, comprising the amino acid sequence of SEQ ID NO: 1.

3. The method according to any one of the preceding claims, wherein the time interval is about three months to about one year.

4. The method according to claim 3, wherein the time interval is about three months to about six months.

5. The method according to claim 3, wherein the time interval is about six months to about one year, preferably wherein the time interval is about six months.

6. The method according to any one of the preceding claims, wherein the drug and hyaluronidase are administered simultaneously or sequentially.

7. The method according to any one of the preceding claims, wherein the micro- or nanoparticles have a surface modifier adsorbed to their surface.

8. The method according to claim 7, wherein the surface modifier is a poloxamer or a polysorbate.

9. The method according to claim 8, wherein the surface modifier is a poloxamer which is poloxamer 338, or wherein the surface modifier is a polysorbate.

10. The method according to any one of the preceding claims, wherein the average effective particle size of the micro- or nanoparticles is less than about 20 μm.

11. The method according to claim 10, wherein the average effective particle size of the micro- or nanoparticles is less than about 10 μm.

12. The method according to claim 11, wherein the average effective particle size of the micro- or nanoparticles is from about 25 nm to about 10 μm.

13. The method according to claim 12, wherein the average effective particle size of the micro- or nanoparticles is from about 200 nm to about 10 μm.

14. The method according to claim 13, wherein the average effective particle size of the micro- or nanoparticles is from about 200 nm to about 5 μm.

15. The method according to any one of the preceding claims, wherein the drug and the hyaluronidase are administered sequentially.

16. The method according to any one of the preceding claims, wherein the drug and the hyaluronidase are administered in separate pharmaceutical compositions.

17. The method according to claim 16, wherein the pharmaceutical composition comprising the hyaluronidase is a solution, and the concentration of the hyaluronidase in the solution is from about 50 to about 10,000 U/mL, preferably about 2,000 U/mL.

18. The method according to any one of claims 1-14, wherein the drug and hyaluronidase are administered as a combined pharmaceutical composition.

19. The method according to any one of the preceding claims, wherein the drug is selected from: drugs for treatment of chronic and long-term diseases and disorders, for example for treatment of chronic viral infection (such as chronic infection with Varicella-zoster virus, measles virus, HIV, hepatitis B virus, hepatitis C virus, hepatitis D virus or human cytomegalovirus), cancer, psychiatric diseases and disorders, mood disorders (such as bipolar, cyclothymic or depression), diabetes, hypertension, abnormal cholesterol and triglyceride levels, inflammatory disorders (such as allergy, asthma, autoimmune diseases, coeliac disease, hepatitis, inflammatory bowel disease, Crohn disease, gout, myositis, scleroderma, rheumatoid arthritis, lupus vasculitis, ankylosing spondylitis or chronic obstructive pulmonary disease), cystic fibrosis, multiple sclerosis, autoimmune disorders, neurodegenerative disorders (such as Parkinson Disease or Alzheimer disease), chronic pain, inherited metabolic disorders or epilepsy

20. The method according to any one of the preceding claims, wherein the drug is selected from rilpivirine, apalutamide, enzalutamide, and darolutamide, or pharmaceutically acceptable salts thereof.

21. The method according to any one of the preceding claims, wherein the drug is rilpivirine or a pharmaceutically acceptable salt thereof.

22. The method according to claim 21, wherein the drug is rilpivirine.

23. The method according to any one of the preceding claims, wherein the drug is not an antibody.

24. The method according to any one of the preceding claims, wherein the drug is not a biologic.

25. The method according to any one of the preceding claims, wherein the drug has a molecular weight of less than 1000 Da.

26. The method according to any one of the preceding claims, wherein the drug and hyaluronidase are administered by subcutaneous injection.

27. The method according to any one of the preceding claims, wherein the suspension comprises a pharmaceutically acceptable aqueous carrier in which the drug is suspended.

28. The method according to any one of the preceding claims, wherein the disease or disorder is HIV infection.

29. The method according to claim 28, wherein the disease or disorder is HIV type 1 (HIV-1) infection.

30. The method according to any one of claims 1-27, wherein the disease or disorder is cancer.

31. The method according to any one of the preceding claims, wherein the subject is a human.

32. A drug and a hyaluronidase for use in therapy,
wherein the drug is in the form of micro- or nanoparticles in suspension,
wherein the drug and hyaluronidase are administered by intramuscular injection or subcutaneous injection, and
wherein the drug and hyaluronidase are administered intermittently at a time interval of about three months to about two years.

33. Products containing a drug and a hyaluronidase as a combined preparation for simultaneous or sequential use in therapy by intramuscular injection or subcutaneous injection,
wherein the drug is in the form of micro- or nanoparticles in suspension, and
wherein the drug and the hyaluronidase are administered intermittently at a time interval of about three months to about two years.

34. A kit of parts comprising a drug and a hyaluronidase for simultaneous or sequential use in therapy by intramuscular injection or subcutaneous injection,
wherein the drug is in the form of micro- or nanoparticles in suspension, and
wherein the drug and the hyaluronidase are administered intermittently at a time interval of about three months to about two years.

35. A drug in the form of micro- or nanoparticles in suspension for use in therapy by intramuscular injection or subcutaneous injection,
wherein the drug is administered in combination with a hyaluronidase that is administered by intramuscular injection or subcutaneous injection, and
wherein the drug and the hyaluronidase are administered intermittently at a time interval of about three months to about two years.

36. Use of a drug for the manufacture of a medicament for use in the treatment of a disease or disorder in a subject,
wherein the drug is in the form of micro- or nanoparticles in suspension and is administered in combination with a hyaluronidase,
wherein the drug and the hyaluronidase are administered to the subject by intramuscular injection or subcutaneous injection, and
wherein the drug and the hyaluronidase are administered intermittently at a time interval of about three months to about two years.


SEQUENCE LISTING (5 sequences)

SEQ ID NO: 1 - protein, 443 aa, Artificial Sequence
  rHuPH20
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEP

SEQ ID NO: 2 - protein, 447 aa, Artificial Sequence [sequence omitted]

SEQ ID NO: 3 - protein, 446 aa, Artificial Sequence
  rHuPH20 variant 2
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIF

SEQ ID NO: 4 - protein, 445 aa, Artificial Sequence
  rHuPH20 variant 3
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQI

SEQ ID NO: 5 - protein, 444 aa, Artificial Sequence
  rHuPH20 variant 4
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQ